(19) United States
(12) Patent Application Publication
(10) Pub. No.: US 2012/0040956 A1
(43) Pub. Date: Feb. 16, 2012

(54) INHIBITORS OF HIF-1 PROTEIN ACCUMULATION

(75) Inventors: Philipp WABNITZ, Dusseldorf (DE); Matthias Gehling, Leichlingen (DE); Thomas Henkel, Wuppertal (DE); M. Lienhard Schmitz, Marburg (DE)

(73) Assignee: INTERMED DISCOVERY GMBH, Dortmund (DE)

(21) Appl. No.: 13/152,365

(22) Filed: Jun. 3, 2011

Related U.S. Application Data
(63) Continuation of application No. PCT/EP2009/008632, filed on Dec. 3, 2009.
(60) Provisional application No. 61/120,102, filed on Dec. 5, 2008.

(30) Foreign Application Priority Data
Dec. 5, 2008 (EP) 08021162.6

(51) Int. Cl. A61K 31/343; A61P 7/00; A61P 13/00; A61P 15/00; A61P 1/00; A61P 17/02; A61P 15/02; A61P 27/02; A61P 27/06; A61P 11/00; A61P 9/12; A61P 13/12; A61P 19/02; A61P 29/00; A61K 31/4025

(52) U.S. Cl. 514/210.19; 514/468; 514/422

(57) ABSTRACT

The invention relates to cyclopentabenzofuran derivatives for the treatment and/or prophylaxis of angiogenesis-related disorders.

This application is a Continuation of PCT/EP2009/008632, filed Dec. 3, 2009, which claims foreign priority benefit under 35 U.S.C. §119 of the European Patent Application No. 08021162.6 filed Dec. 5, 2008 and U.S. Provisional Patent Application No. 61/120,102 filed Dec. 5, 2008, the disclosures of which are incorporated herein by reference.

The invention relates to cyclopentabenzofurane derivatives useful for the treatment and/or prophylaxis of angiogenesis-related disorders, preferably pulmonary hypertension.

Hypoxia-inducible factor 1 (HIF-1) is a transcription factor that regulates the expression of several genes involved in key aspects of adaptive responses to hypoxia, including cellular immortalization, maintenance of stem cell pools, cellular de-differentiation, erythropoiesis, genetic instability, vascularisation, metabolic reprogramming, autocrine growth factor signaling, and invasion/metastasis.

The HIF-1 transcription factor is formed as a heterodimer by oxygen-regulated HIF-1α and constitutively expressed HIF-1β. The latter does also dimerize with the structurally and functionally related HIF-2α protein regulating an overlapping battery of target genes. The HIF-1 complex mediates expression of many genes such as e.g. VEGF, EPO, LDH, PDK1 etc. which are considered to be key mediators within the above mentioned biological processes.

The transcriptional activity of HIF-1 is closely controlled by a hypoxic stimulus. Under normoxic (˜20% O₂) conditions, HIF-1α (as well as HIF-2α) as a subject to oxygen mediated prolyl hydroxylation by Prolyl Hydroxylase (PHD) underlies a high turn-over (half-life: ca. 5 min). The modification by PHD is required for binding of the von Hippel-Lindau tumor suppressor protein (VHL), which also binds to Elongin C and thereby recruits an ubiquitin ligase complex that targets HIF-1α for ubiquitination. The ubiquitinated HIF-1α protein is then degraded by the proteasomal complex. In contrast, the rate of hydroxylation and ubiquitination declines under hypoxic conditions, resulting in accumulation and stabilization by e.g. nitrosylation of HIF-1α (see FIG. 1a: schematic drawing of HIF-1 protein mediated (pro-angiogenic) activity due to hypoxic or VEGF stimuli).

Specific modulation of HIF-1 protein dependent transcription will allow specific modulation/treatment of vascularisation and vascular remodelling. Pathological vascularisation and vascular remodelling are associated with multiple human disorders such as e.g. cancer (i.e. tumor vascularisation) or pulmonary hypertension and can be induced by e.g. lack of oxygen (hypoxia; compare FIG. 1a). HIF-1 activity is induced in response to continuous hypoxia, intermittent hypoxia, growth factor stimulation and mediates e.g. maladaptive responses to chronic continuous and intermittent hypoxia, which underlie the development of pulmonary and systemic hypertension (Semenza G L. Physiology (Bethesda), 2009; 24:97-106). Very recently HIF-1, EPO and VEGF activity have been associated with pulmonary hypertension in infants (Lemus-Varela M L et al., Expression of HIF-1alpha, VEGF and EPO in peripheral blood from patients with two cardiac abnormalities associated with hypoxia. Clin Biochem. 2009).

HIF-1 has been reported to be regulated oxygen-dependently thereby mediating the adaptive response to changes in tissue oxygenation, see J. J. Haddad, Oxygen-sensing mechanisms and the regulation of redox-responsive transcription factors in development and pathophysiology. Respir Res 2002, 3:26 and G. Semenza, Targeting HIF-1 for Cancer Therapy. NatRevCancer 2003, 3: 721-732.

HIF-1 has also been reported to stimulate transcriptional activation of vascular endothelial growth factor (VEGF), a ligand of the VEGF receptor family which in turn stimulates cellular proliferation and angiogenesis. See J. M. G. Larkin and T. Eisen, Renal cell carcinoma and the use of Sorafenib. 2006, Therapeutics and Clinical Risk Management, 2(1): 87-98. Suppression and loss-of-function of HIF-1 have been reported to be associated with reduced tumor growth, vascularisation and metastasis, see G. Semenza, Evaluation of HIF-1 inhibitors as anticancer agents. Drug Discovery Today 2007, 12(19/20): 853-859

HIF-1 overexpression has also been observed in animal models in association with tumor growth, increased vascularisation, and metastasis. Most of locally advanced solid tumors contain regions of reduced oxygen availability. This intratumoral hypoxia results out of the tumor cells distance from a functional blood vessel which hinders the diffusion of adequate amounts of oxygen as a result of rapid cancer cell proliferation and disturbed formation of blood vessels. In the meantime, immunohistochemical detection of HIF-1α overexpression in biopsy sections has become a prognostic factor in many cancers. A growing number of novel anticancer agents have been shown to inhibit HIF-1 through a variety of molecular mechanisms (Semenza G. L.; 2007, Drug Discovery Today, Vol. 12, 19/20, 853-859).

Determining which combination of drugs to administer to any given patient remains a major obstacle to improving cancer treatment outcomes.

In contrast to these findings, inhibition of HIF-1 mediated transcription has been shown to have the opposite effect, thus validating HIF-1 as a target for treatment of hypoxia and angiogenesis (neo-vascularization) related disorders.

Since HIF-1 action is located more downstream within a regulatory pathway triggered by e.g. hypoxia and other stimuli such as e.g. VEGF-receptor signal, the point of intervention targeted by compounds of this invention will result in much more specific effects compared to e.g. marketed VEGF-receptor inhibitors such as e.g. Sunitinib; Sorafenib and Avastin (see FIG. 1b). Furthermore compounds of this invention will be able to influence or modify hypoxia induced physiological signaling more directly and precisely than e.g. VEGF or other Receptor Tyrosine Kinase (RTK) inhibitors. Thus using compounds of this invention novel therapeutic approaches can be designed, therapies can be optimized (personalized) by e.g. combinatory therapeutic approaches and side effects can be reduced. Furthermore compounds of this invention will show superior effects compared to biological entities such as antibodies, since the pharmacologically useful action of the compounds of this invention are able to penetrate cellular membranes. Therefore compounds of this invention are able to take their effect inside of mammalian cells while anti-body based effectors will usually not be able to even reach the cells' interior.

More specific diseases are also know from the art as to be dependent of HIF-1 expression as well as the production of the related Hif protein:

Endometriosis does mean the presence of ectopic endometrial tissue outside the uterine cavity. E. is a common disease affecting women during their reproductive years.

Hif-1 has been reported to have a role in the regulation of endometriosis, see Becker et al., 2-Methoxyestradiol Inhibits Hypoxia-Inducible Factor-1alpha and Suppresses Growth of Lesions in a Mouse Model of Endometriosis. Am J Pathol 2008, 172:534-544. Inhibitors of VEGF and/or HIF-1 (signal) as a mediator of VEGF expression has been described in the art as potential therapeutic approach to treat pulmonary disorders such as e.g. chronic obstructive pulmonary disease (COPD) and pulmonary hypertension, see H. Kanazawa, Role of vascular endothelial growth factor in the pathogenesis of chronic obstructive pulmonary disease. MedSciMonit 2007, 13(11): RA189-195.

In the art HIF-1 has been described to be associated with inflammatory processes via hypoxia and angiotensin receptor expression, see G. R. Smith, Cancer, inflammation and the AT1 and AT2 receptors. Journal of Inflammation 2004, 1:3.

HIF-1 has been described in the art as target for therapeutic approaches towards hypoxia-induced kidney fibrosis and ESRD, see M. Nangaku et al., Role of chronic hypoxia and hypoxia inducible factor in kidney Disease. Chinese Medical Journal 2008; 121(3):257-264 257.

Up-regulation of HIF-1 has been described in the art to be associated with peyronie's disease, see M. Lucattelli et al., A new mouse model of Peyronie's disease: an increased expression of hypoxia-inducible factor-1 target genes during the development of penile changes. Int J Biochem Cell Biol. 2008, 40(11):2638-48.

Hif-1 alpha overexpression has been associated in the art with erectile dysfunction, see M. Lee et al., Efficient gene expression system using the RTP801 promoter in the corpus cavernosum of high-cholesterol diet-induced erectile dysfunction rats for gene therapy. J Sex Med. 2008 June; 5(6):1355-64.

In the art HIF-1 overexpression has been described to promote fibrosis, see V. H. Haase, Pathophysiological Consequences of HIF Activation: HIF as a modulator of fibrosis. Ann NY Acad. Sci. 2009, 1177:57-65.

In the arts hypoxia-induced HIF-1 has been described as contributor to the progression of scleroderma, see K. H. Hong et al., Hypoxia induces expression of connective tissue growth factor in scleroderma skin fibroblasts. Clin Exp Immunol. 2006, 146(2):362-70.

HIF-1 overexpression due to hypoxia has been clinically associated with ARDS progression, see N. Hirani, The regulation of interleukin-8 by hypoxia in human macrophages—a potential role in the pathogenesis of the acute respiratory distress syndrome (ARDS). Mol. Med. 2001, 7(10):685-97.

In the arts HIF-1 has been associated with atherosclerosis; see N. Adhikari et al., Transcription factor and kinase-mediated signaling in atherosclerosis and vascular injury. Curr Atheroscler Rep. 2006, 8(3):252-60 and J. C. Sluimer and M. J. Daemen, Novel concepts in atherogenesis: angiogenesis and hypoxia in atherosclerosis. J Pathol. 2009, 218(1):7-29. HIF-1 expression has been associated with hemangioblastoma, see D. Zagzag et al., Expression of hypoxia-inducible factor 1alpha in brain tumors: association with angiogenesis, invasion, and progression. Cancer. 2000, 88(11):2606-18 and M. Krieg et al., Up-regulation of hypoxia-inducible factors HIF-1alpha and HIF-2alpha under normoxic conditions in renal carcinoma cells by von Hippel-Lindau tumor suppressor gene loss of function. Oncogene. 2000, 19(48):5435-43.

In the arts HIF-1 expression has been described a contributing factor for a metastatic phenotype of tumor cells, see N. Simiantonaki et al., Hypoxia-inducible factor 1 alpha expression increases during colorectal carcinogenesis and tumor progression. BMC Cancer. 2008, 8:320.

HIF-1 has been described in the arts as therapeutic target for the treatment of macular degeneration, see O. Arjamaa O, Regulatory role of HIF-1alpha in the pathogenesis of age-related macular degeneration (AMD). Ageing Res Rev. 2009, 8(4):349-58.

There are known angiogenesis inhibitors already in used as pharmaceuticals in humans. The use of Sorafenib (Nexavar®, Trademark by Bayer HealthCare) has been described in the art as kinase inhibitor that significantly reduces angiogenesis (e.g. tumor vascularisation) by inhibiting the vascular endothelial growth factor (VEGF) receptor, amongst others. See J. M. G. Larkin and T. Eisen, Renal cell carcinoma and the use of Sorafenib. 2006, Therapeutics and Clinical Risk Management, 2(1): 87-98.

Sunitinib (Sutent®, Trademark by Pfizer; formerly distributed as SU11248) has been described in the art as anti-angiogenic effector that exhibits direct antitumor and anti-angiogenic activity via inhibition of the receptor tyrosine kinases platelet-derived growth factor receptor, vascular endothelial growth factor receptor, KIT, and FLT3, see P. Marzola P et al., Early anti-angiogenic activity of SU11248 evaluated in vivo by dynamic contrast-enhanced magnetic resonance imaging in an experimental model of colon carcinoma. Clin Cancer Res 2005, 11(16): 5827-32 and S. de Boüard et al., Anti-angiogenic and anti-invasive effects of sunitinib on experimental human glioblastoma. Neuro Oncol. 2007, 9(4):412-23. PTK787/ZK 222584 (Vatalanib, collaboration Novartis and Bayer Schering A G) has previously been reported to act as an anti-angiogenic VEGF receptor inhibitor, see A. Alajati et al., Spheroid-based engineering of a human vasculature in mice. Nat Methods 2008, 5, 439-445.

Other VEGF receptor inhibitors are Vandetanib (Zactima®, Trademark by AstraZeneca; formerly distributed as ZD6474), AZD2171 (Recentin®, Trademark by AstraZeneca) and the anti-body Bevacizumab (Avastin®, Trademark by Genentech/Roche).

However, the properties of the known VEGF and RTK inhibitors used as HIF inhibitors are not satisfactory in every respect and thus, there is a demand for further HIF inhibitors, especially for HIF-1 inhibitors being useful for the treatment of hypoxia and angiogenesis (neo-vascularisation) related disorders.

It is an object of the invention to provide compounds that have advantages over the compounds of the prior art. The compounds should effectively inhibit HIF at comparatively low doses and should be useful for the treatment and/or prophylaxis of angiogenesis-related disorders.

This object has been solved by the subject-matter of the patent claims.

It has been surprisingly found that certain cyclopentabenzofuranes exhibit HIF inhibitory activity. These cyclopentabenzofuranes can be i.e. derived from a class of natural products which are referred to as rocaglaols or rocaglamides which can be i.e. extracted from various species of the Aglaia plant.

A number of cyclopentabenzofurane derivatives is known from the prior art that i.e. exhibit an inhibitory activity against the NF-KB transcription factor which occupies a central role in inflammatory processes and carcinogenesis. For example, cyclopentabenzofuranes are known as potent anticancer agents (King, M. A. et al., J. Chem. Soc., Chem. Commun. 1982: 1150-1151), i.e. as anti-leukaemia agents (Lee, S. K. et al., Chem. Biol. Interaet. 1998, 115: 215-228; U.S. Pat. No. 4,539,414). Furthermore, cyclopentabenzofurane derivatives are also known to be useful for the treatment of pain (WO 2008/014066) and for the treatment of inflammatory and/or autoimmune diseases (EP 1 693 059; WO 2005/113529; WO 2006/129318).

WO 01/12592 discloses hydroxamic acid compounds useful as matrix metalloproteinase inhibitors.

EP 1 016 408 relates to the use of certain C—C chemokine production inhibitors for manufacture of a medicament for certain disorders including chronic intractable inflammation and chronic rheumatoid arthritis.

Compounds of this invention have demonstrated very potent (low nM) inhibition of HIF-1 dependent luciferase transcription and single digit nanomolar inhibition of HIF-1 dependent target gene transcription (e.g. PDK1). It was demonstrated that these effects were not mediated on the transcriptional (mRNA) level and can be reached in a therapeutic manner (compound application after signal induction). Furthermore it was demonstrated that the HIF-dependent effects were triggered by a potent inhibition of HIF-1α protein itself. This effect was shown to be Hif-1a protein specific and not a result of un-specific inhibition of translational processes. In vitro the compounds of this invention did show a potent inhibition of HUVEC sprouting (angiogenesis; vascular modeling) at two-digit nanomolar IC₅₀s. In this in vitro model the compounds performed better than marketed benchmark compounds such as Sunitinib (Sutent®) and Sorafenib (Nexavar®). In VEGF induced HUVEC sprouting assays compounds of this invention did reach IC₅₀ values better (lower) than Sutent® by a factor of 2.5 to 6 times and better than Nexavar® by a factor of 50 to 100 times. These values improved even more when HUVECs were induced by Deferoxamine (hypoxia). In these cases IC₅₀ of the compounds of invention were better than Sutent® by a factor of 33-100 while Nexavar® did not show any activity at all. These findings demonstrate that hypoxic and VEGF stimulation (pathways; see also FIGS. 1a and b) share some features but have also significant differences, substantiating that compounds of this invention will enable a completely new access towards treatment of e.g. neovascularisation and/or vascular remodeling than e.g. VEGF receptor inhibitors such as e.g. Sunitinib (Sutent®) and Sorafenib (Nexavar®).

Common side-effects of e.g. such VEGF/Receptor Tyrosine Kinase inhibitors or other upstream Modulators of HIF-1 signaling such as e.g. diarrhea, eczema, hair loss, hemorrhage, hypertension, hypothyroidism, nausea, emesis, erythema, itchiness, fatigue, pain and increased amylase and lipase activity.

However, cyclopentabenzofuranes that exhibit HIF inhibitory activity, have not been reported in the literature.

### BRIEF DESCRIPTION OF THE DRAWINGS

The invention will now be described in greater detail with reference to the drawings, wherein:

FIG. 1a is a schematic drawing of HIF-1 protein mediated (pro-angiogenic) activity due to hypoxic or VEGF stimuli;

FIG. 1b is a schematic drawing of HIF-1 action due to hypoxic or VEGF stimuli;

FIG. 2a is a graph showing IMD-compound influence on HIF-1 mediated luciferase activity in 239T cells;

FIG. 2b is a graph showing IMD-compound influence on HIF-1 mediated luciferase activity in Jurkat T cells;

FIG. 3 is a series of graphs showing the influence of different IMD-compounds on HIF-1 mediated luciferase activity in cell extracts;

FIG. 4 is a graph showing IMD-compound influence on HIF-1 mediated luciferase activity in 239T cells;

FIG. 5 is a graph showing effect of IMD-compounds on HIF-1-dependent gene expression;

FIG. 6a is a graph showing two independent experiments confirming PDK1 as the gene which was induced most prominently compared to LDH;

FIG. 6b is a series of graphs showing the results of an experiment in which cells were incubated under normoxic/hypoxic conditions for further 8 h and the expression of PDK1 was quantified by real-time PCR;

FIG. 7 is a gel proving the accumulation of HIF-1α protein was not detectable at normoxic conditions (−) while being induced by hypoxia (+);

FIG. 8 is a schematic showing an assay principle;

FIGS. 9a and 9b are a set of gels showing the formed proteins analyzed by SDS-PAGE from Brome mosaic virus (BMV) mRNA encoding for 4 different viral proteins incubated with ribosomes in the presence and absence of IMD-019064 (FIG. 9a), IMD-026259, IMD-026260 (FIG. 9b), or cycloheximide (Chx, 10 μM) (positive control);

FIG. 10 is a gel showing proteins separated by SDS-PAGE after non-radioactive in vitro translation of Flag-tagged NF-κB p50 protein and detecting the produced p50 protein by immunoblotting using Flag antibodies;

FIG. 11 constitutes a schematic of an assay; and a chart showing the results, indicating that inhibitory activity of IMD-019064 on cellular protein synthesis is mediated by specifically blocking e.g. IL-1 dependent signaling cascades;

FIG. 12 is a gel showing protein contained in the cell lysates used for immunoblotting with anti-IKKy/NEMO antibodies FIGS. 13a and 13b are a series of graphs depicting EC₅₀ and fibroblast scattering rate compared to control for various IMD-compounds;

FIG. 14a is a series of graphs depicting EC spouting rate compared to control for various IMD-compounds;

FIG. 14b is a table summarizing results of IMD-compound testing;

FIG. 15 is a graph showing IMD-026260 reduction of human vessel number compared to a vehicle control; and

FIG. 16 is a schematic showing repetitive hypoxia vasoconstriction.

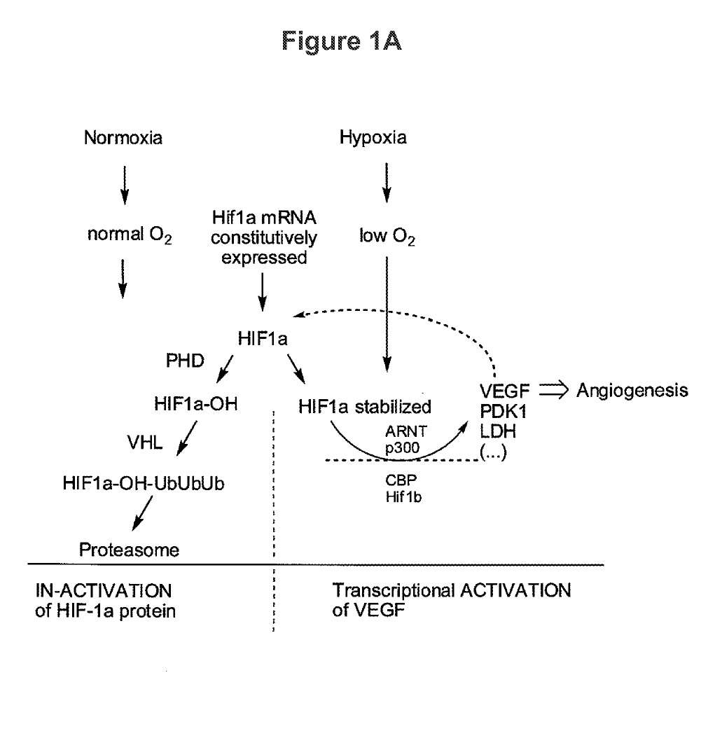

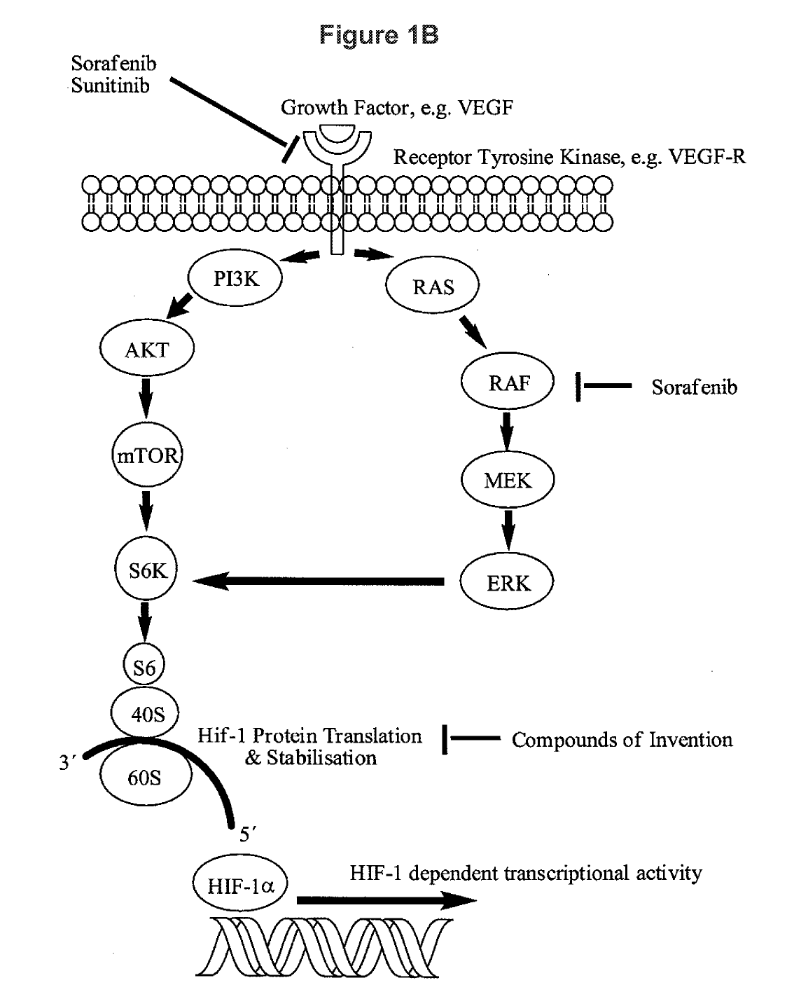

A first aspect of the invention relates to compounds of general formula (I)

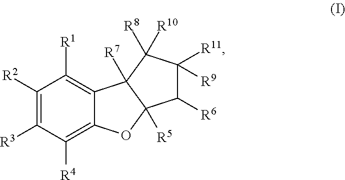

wherein
R¹, R², R³ and R⁴ independently of each other denote —H; —F; —Cl; —Br; —I; —NO₂; —CN; —OH; —O—C₁₋₈-alkyl; —O-phenyl; —O—C₁₋₈-alkyl-phenyl; —O—C(═O)—C₁₋₈-alkyl; —O—C(═O)-phenyl; carbohydrate bound via one of its oxygen atoms; 6-(1,2-dihydroxy-ethyl)-3-methoxy-2-hydroxy-1,4-dioxan-2-yl;
or R¹ and R² or R² and R³ or R³ and R⁴ together with the two carbon atoms they are bound to form a five-membered ring with —O—CH₂—O— or a six-membered ring with —O—CH₂—CH₂—O—, while the other radicals R¹ to R⁴ are independently selected from those mentioned above;
R⁵ and R⁶ are phenyl;
R⁷ is —OH; —O—C₁₋₁₂-alkyl; —O-phenyl; —O—C₁₋₈-alkyl-phenyl; —O—C(═O)—C₁₋₈-alkyl; —O—C(═O)-phenyl;
R⁸ is —H; —OH; —O—C₁₋₈-alkyl; —O-phenyl; —NH₂; —NH—C₁₋₈-alkyl; —N(C₁₋₈-alkyl)₂;
R⁹ is —H; —C(═O)—OH; —C(═O)—O—C₁₋₈-alkyl; —C(═O)—O-phenyl; —C(═O)—C₁₋₈-alkyl; —C(═O)—O—C₁₋₈-alkyl; —C(═O)—NH₂; —C(═O)—NH—C₁₋₈-alkyl; —C(═O)—N—(C₁₋₈-alkyl)₂; or denotes —C(═O)-heterocyclyl, wherein said heterocyclyl contains at least one N-atom which is bound to the C(═O)-group;

### R¹⁹ and R¹¹ are —H;

or R⁸ and R¹⁹ together denote ═O, ═S, or ═NR¹⁵,
wherein R¹⁵ is —C₁₋₈-alkyl; —OH; —O—C₁₋₈-alkyl; or —O-phenyl;
   or R¹⁰ and R¹¹ together form a single bond and R⁸ and R⁹ together form a group of the formula (II)

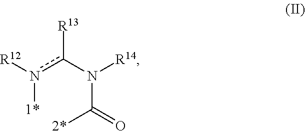

wherein
1* is the bond via R⁸ and
2* is the bond via R⁹, respectively;
the dotted line is a single or a double bond, wherein in case of a double bond R¹² is not existing;
R¹² is —H or —C₁₋₃-alkyl;
R¹³ is —H; —OH; —O—C₁₋₈-alkyl; or —O-phenyl;
R¹⁴ is —H, —C₁₋₈-alkyl;
or R¹³ and R¹⁴ together with the carbon and nitrogen atoms they are bound to form a heterocyclyl;
   wherein “alkyl” in each case can be unsubstituted or substituted with one, two or three substituents independently of each other selected from the group consisting of —F, —Cl, —Br, —I, —OH, —OCH₃, —OCH₂CH₃, —O—CH₂-phenyl, —OC(═O)CH₃, —CHO, —CO₂H, —NH₂, —NH—(C₁₋₈-alkyl), —NH-(phenyl), —NH—(CH₂-phenyl), —N(C₁₋₈-alkyl)₂ and heterocyclyl, wherein said heterocyclyl contains at least one N-atom which is connected to the alkyl residue.
   wherein “phenyl” in each case can be unsubstituted, or substituted with one, two or three substituents independently of each other selected from the group consisting of —F, —Cl, —Br, —I, —OH, —OCH₃, —OCH₂CH₃, —OC(═O)CH₃, —CN, —NO₂, —NH₂, —CH₃, CF₃, —CHO and —CO₂H;
   or the physiologically acceptable salts thereof,
   for the treatment and/or prophylaxis of angiogenesis-related disorders, preferably selected from the group consisting of diseases of the urogenital tract, eye diseases, lung diseases, kidney diseases, osteoarthritis and rheumatic disorders; particularly preferably pulmonary hypertension.

For the purpose of the specification, “alkyl” or “C₁₋₈-alkyl” refers to a saturated or unsaturated, linear or branched and/or cyclic hydrocarbon. Thus, the term “alkyl” encompasses “alkyl”, “alkenyl” and “alkynyl” as well as “cycloalkyl”, “cycloalkenyl” and “cycloalkynyl”. Examples of preferred alkyl residues are methyl, ethyl, n-propyl, i-propyl, n-butyl, sec.-butyl, iso.-butyl, tert.-butyl, n-pentyl, n-hexyl, n-heptyl and n-octyl. Examples of preferred alkenyl residues include vinyl, allyl and butadienyl. Examples of preferred alkynyl residues include ethynyl and propargyl. A skilled person recognizes that a cyclic hydrocarbon requires the presence of at least 3 ring atoms. Examples of preferred cycloalkyl residues are cyclopropyl, cyclobutyl, cyclopentyl and cyclohexyl. An alkyl residue can be unsubstituted or substituted with one, two or three substituents independently of each other selected from the group consisting of —F, —Cl, —Br, —I, —OH, —OCH₃, —OCH₂CH₃, —O—CH₂-phenyl, —OC(═O)CH₃, —CHO and —CO₂H, —NH₂, —NH—(C₁₋₈-alkyl), —NH-(phenyl), —NH—(CH₂-phenyl), —N(C₁₋₈-alkyl)₂, and heterocyclyl, wherein said heterocyclyl contains at least one N-atom which is connected to the alkyl residue and is preferably selected from the group consisting of aziridinyl, azetidinyl, pyrrolidinyl and piperidinyl. Preferably, —NH(C₁₋₈-alkyl) is selected from the group consisting of —NH(CH₃), —NH(CH₂CH₃) and —NH(CH(CH₃)₂). Preferably, —N(C₁₋₈-alkyl)₂ is selected from the group consisting of —N(CH₃)₂, —N(CH₂CH₃)₂, N(CH(CH₃)₂)₂ and —N(C₃H₇)₂. Examples of preferred substituted alkyl residues are —CF₃, —CH₂CH₂—OCH₃, —CH₂CH₂NH₂, —CH₂CH₂—NH(CH₃), —CH₂CH₂—N(CH₃)₂, —CH₂CH₂—NH(CH₂CH₃), —CH₂CH₂—N(CH₂CH₃)₂, —CH₂—CH₂-pyrrolidinyl, —CH₂CH₂CH₂-pyrrolidinyl, —CH₂CH₂CH₂-azetidinyl and —CH₂CH₂CH₂-aziridinyl.

For the purpose of the specification, “phenyl” refers to a benzene moiety, unsubstituted or substituted with one, two or three substituents independently of each other selected from the group consisting of —F, —Cl, —Br, —I, —OH, —OCH₃, —OCH₂CH₃, —OC(═O)CH₃, —CN, —NO₂, —NH₂, —CH₃, —CF₃, —CHO and —CO₂H; such as phenyl, o-fluorophenyl, m-fluorophenyl, p-fluoro-phenyl, o-chlorophenyl, m-chlorophenyl, p-chlorophenyl, o-anisyl, m-anisyl, p-anisyl, and the like.

For the purpose of the specification, “heterocyclyl” or “heterocycle” refer to a saturated, unsaturated, or aromatic three- to seven-membered ring, i.e. three-, four-, five-, six-, or seven-membered ring containing one or two heteroatoms, preferably one heteroatom selected from the group consisting of N, O and S, preferably N, and wherein said ring is unsubstituted or contains one or two substituents selected from the group consisting of —OCH₃, —OCH₂CH₃, —OC(═O)CH₃, —OC(═O)CH₂CH₃, —OC(═O)CH₂(CH₃)₂, —NHC(═O)CH₃, —NHC(═O)CH₂CH₃, —NHC(═O)CH(CH₃)₂. Examples of preferred heterocyclyls or heterocycles are aziridinyl, azetidinyl, pyrrolidinyl, imidazolyl, piperidinyl, morpholinyl and pyridinyl.

For the purpose of the specification, “carbohydrate” or “C₅₋₁₂-carbohydrate” refer to a mono or disaccharide consisting of one or two pentoses (C₅-carbohydrate or C₁₋₁₀-carbohydrate) and/or one or two hexoses (C₆-carbohydrate or C₁₋₂-carbohydrate), each optionally in their desoxy forms, the disaccharides in each form connected to each other via a glycosidic bond, unsubstituted or substituted with one, two, three, four or five substituents independently selected from the group consisting of methyl, ethyl, acetyl, benzoyl or 3,4,5-trihydroxy-benzoyl. Examples of preferred pentoses are xylose, arabinose, each in the pyranosidic or furanosidic form. Examples of preferred hexoses are glucose, 6-deoxyglucose, rhamnose, each in the pyranosidic of furanosidic form. Examples of preferred glycosidic connections are 1→4 and 1→6. “Carbohydrate” or “C₅₋₁₂-carbohydrate” residues are bound to the higher general formula via one of its oxygen atoms.

Physiologically acceptable salts of the compounds of general formula (I) include salts with physiologically acceptable acids as well as salts with physiologically acceptable bases. Physiologically acceptable acids include inorganic acids such as HCl, HBr, H₂SO₄, H₃PO₄ and the like; and organic acids such as formic acid, acetic acid, propionic acid, citric acid, maleic acid, malic acid, lactic acid, fumaric acid, and the like. Physiologically acceptable bases include ammonia, and organic amines.

The invention also relates to the stereoisomers of the compounds of general formula (I), such as enantiomers or diastereomers, tautomeric forms, salts, solvates such as hydrates, polymorphs, and the like.

In a preferred embodiment of the compounds according to the general formula (I),

R¹, R², R³ and R⁴ independently of each other denote —H; —F; —Cl; —Br; —I; —NO₂; —CN; —OH; —O—C₁₋₈-alkyl; —O-phenyl; —O—C₁₋₈-alkyl-phenyl; —O—C(═O)—C₁₋₈-alkyl; —O—C(═O)-phenyl; —C₅₋₁₂-carbohydrate bound via one of its oxygen atoms; 6-(1,2-dihydroxy-ethyl)-3-methoxy-2-hydroxy-1,4-dioxan-2-yl;
or R¹ and R² or R² and R³ or R³ and R⁴ together with the two carbon atoms they are bound to form a five-membered ring with —O—CH₂—O— or a six-membered ring with —O—CH₂—CH₂—O—, while the other radicals R¹ to R⁴ are independently selected from those mentioned above;
R⁸ is H; —OH; —O—C₁₋₈-alkyl; —O-phenyl; —NH₂; —NH—C₁₋₈-alkyl; —N(C₁₋₈-alkyl)₂;
R⁹ is H; —C(═O)—OH; —C(═O)—O—C₁₋₈-alkyl; —C(═O)—O-phenyl; —C(═O)—C₁₋₈-alkyl; —C(═O)—O—C₁₋₈-alkyl; —C(═O)—NH₂; —C(═O)—NH—C₁₋₈-alkyl; —C(═O)—N—(C₁₋₈-alkyl)₂; or denotes —C(═O)-heterocyclyl, wherein said heterocyclyl contains at least one N-atom which is bound to the C(═O)-group;

### R¹⁰ and R¹¹ are —H;

or R⁸ and R¹⁹ together denote ═O, ═S, or ═NR¹⁵,
wherein R¹⁵ is —C₁₋₈-alkyl; —OH; —O—C₁₋₈-alkyl; or —O-phenyl.

Preferred compounds of general formula (I) are of general formulae (Ia), (Ib), (Ic) or (Id):

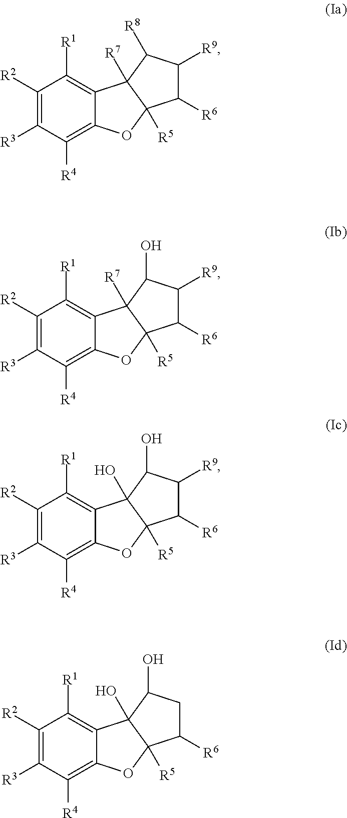

In a preferred embodiment of the compounds according to one of the general formulae (I) or (Ia),

R¹, R², R³ and R⁴ independently of each other denote —H; —F; —Cl; —Br; —I; —NO₂; —CN; —OH; —O—C₁₋₈-alkyl; —O-phenyl; —O—C₁₋₈-alkyl-phenyl; —O—C(═O)—C₁₋₈-alkyl; —O—C(═O)-phenyl;
R⁵ and R⁶ are phenyl;
R⁷ and R⁸ are independently of each other —OH, or —O—C₁₋₈-alkyl;

### R⁹, R¹⁰ and R¹¹ are —H.

In another preferred embodiment of the compounds according to one of the general formulae (I), (Ia) or (Ib)

R¹, R², R³ and R⁴ independently of each other denote —H; —F; —Cl; —Br; —I; —NO₂; —CN; —OH; —O—C₁₋₈-alkyl; —O-phenyl; —O—C₁₋₈-alkyl-phenyl; —O—C(═O)—C₁₋₈-alkyl; —O—C(═O)-phenyl;
R⁵ and R⁶ are phenyl;
R⁷ is —OH, or —O—C₁₋₈-alkyl;

### R⁸ is OH;

### R⁹, R¹⁰ and R¹¹ are —H.

In another preferred embodiment of the compounds according to one of the general formulae (I), (Ia), (Ib), (Ic) or (Id)

R¹, R², R³ and R⁴ independently of each other denote —H; —F; —Cl; —Br; —I; —NO₂; —CN; —OH; —O—C₁₋₈-alkyl; —O-phenyl; —O—C₁₋₈-alkyl-phenyl; —O—C(═O)—C₁₋₈-alkyl; —O—C(═O)-phenyl;
R⁵ and R⁶ are phenyl;

### R⁷ and R⁸ are OH;

### R⁹, R¹⁰ and R¹¹ are —H.

In another preferred embodiment of the compounds according to the general formula (I),

R¹ and R³ independently of each other denote —H; —OH; —O—C₁₋₈-alkyl; —O-phenyl; —O—C₁₋₈-alkyl-phenyl; —O—C(═O)—C₁₋₈-alkyl; —O—C(═O)-phenyl;
with the proviso that at least one of the radicals R¹ and R³ is not —H;

### R² and R⁴ are H;

R⁵ and R⁶ are phenyl,
R⁷ and R⁸ are independently of each other —OH, or —O—C₁₋₈-alkyl;

### R⁹, R¹⁰ and R¹¹ are —H.

In yet another preferred embodiment of the compounds according to one of the general formulae (I), (Ia), (Ib), (Ic) or (Id)

R¹ and R³ independently of each other denote —H; —OH; —O—C₁₋₈-alkyl; —O-phenyl; —O—C₁₋₈-alkyl-phenyl; —O—C(═O)—C₁₋₈-alkyl; —O—C(═O)-phenyl;
with the proviso that at least one of the radicals R¹ and R³ is not —H;

### R² and R⁴ are —H;

R⁵ and R⁶ are phenyl,
R⁷ and R⁸ denote —OH;

### R⁹, R¹⁰ and R¹¹ are —H.

In another preferred embodiment of the compounds according to one of the general formulae (I), (Ia), (Ib), (Ic) or (Id)

R¹ and R³ independently of each other denote —H; —OH; —O—C₁₋₈-alkyl; O-phenyl; —O—C₁₋₈-alkyl-phenyl;
with the proviso that at least one of the radicals R¹ and R³ is not —H;

### R² and R⁴ are —H;

R⁵ and R⁶ are phenyl,

### R⁷ and R⁸ are —OH;

### R⁹, R¹⁹ and R¹¹ are —H.

Another preferred compound of general formula (I) is of general formula (Ie):

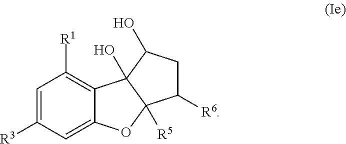

In a preferred embodiment of the compounds according to the general formula (Ie)

R¹ denotes H; unsubstituted or substituted with one substituent selected from the group consisting of —OCH₃, —OCH₂CH₃, —NH₂, —NH(CH₃), —NH(CH₂CH₃), —N(CH₃)₂, —N(CH₂CH₃)₂,

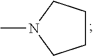

—O—CH₂-phenyl, unsubstituted; preferably —OCH₂CH₂— substituted with —N(CH₃)₂ or

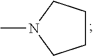

R³ denotes —OH; —O-phenyl, unsubstituted; —O—C₁₋₈-alkyl, unsubstituted or substituted with one substituent selected from the group consisting of —F, —Cl, —OCH₃, —OCH₂CH₃, —NH₂, —NH(CH₃), —NH(CH₂CH₃), —NH(CH(CH₃)₂), —NH(C(CH₃)₃), —NH(CH₂-phenyl) or —NH(phenyl), wherein phenyl in each case is unsubstituted, —N(CH₃)₂, —N(CH₂CH₃)₂,

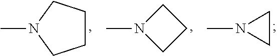

—O—CH₂-phenyl, unsubstituted;

R⁵ and R⁶ are phenyl, unsubstituted, or substituted with one, two or three substituents independently of each other selected from the group consisting of —F, —Cl, —Br, —I, —OH, —OCH₃, —NH₂, —CH₃ and —CF₃.

Particularly preferred compounds according to the invention and according to general formula (If) are summarized in the table (Table 1) here below:

TABLE 1
(If)

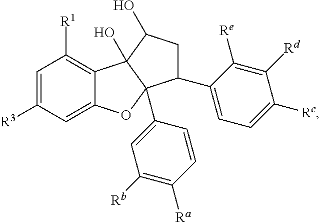

| no. | R¹ | R³ | R^{a} | R^{b} | R^{c} | R^{d} | R^{e} |
|---|---|---|---|---|---|---|---|
| 1 | —H | —OH | —Cl | —H | —H | —H | —H |
| 2 | —H | —OCH₃ | —H | —H | —H | —H | —H |
| 3 | —H | —OCH₃ | —Cl | —H | —H | —H | —H |
| 4 | —H | —OCH₃ | —Br | —H | —H | —H | —H |
| 5 | —H | —OCH₂CH₃ | —Cl | —H | —H | —H | —H |
| 6 | —H | —OCH₂CH₃ | —Br | —H | —H | —H | —H |
| 7 | —H | —OCH₂Ph | —Cl | —H | —H | —H | —H |
| 8 | —H | —OPh | —Cl | —H | —H | —H | —H |
| 9 | —H | —O(CH₂)₂—O—CH₃ | —Cl | —H | —H | —H | —H |
| 10 | —H | —O(CH₂)₂—NH₂ | —Cl | —H | —H | —H | —H |
| 11 | —H | —O(CH₂)₂—NH—CH₃ | —H | —H | —H | —H | —H |
| 12 | —H | —O(CH₂)₂—NH—CH₃ | —Cl | —H | —H | —H | —H |
| 13 | —H | —O(CH₂)₂—N—(CH₃)₂ | —Cl | —H | —H | —H | —H |
| 14 | —H | —O(CH₂)₂—NH—CH₂CH₃ | —Cl | —H | —H | —H | —H |
| 15 | —H | —O(CH₂)₂—N—(CH₂CH₃)₂ | —Cl | —H | —H | —H | —H |
| 16 | —H | —O(CH₂)₂—NH—CH(CH₃)₂ | —Cl | —H | —H | —H | —H |
| 17 | —H | —O(CH₂)₂—NH—C(CH₃)₃ | —Cl | —H | —H | —H | —H |
| 18 | —H | 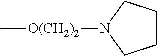 | —Cl | —H | —H | —H | —H |
| 19 | —H | —O(CH₂)₂—NH—CH₂Ph | —Cl | —H | —H | —H | —H |
| 20 | —H | —O(CH₂)₂—NH—Ph | —Cl | —H | —H | —H | —H |
| 21 | —H | 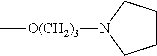 | —Cl | —H | —H | —H | —H |
| 22 | —H | 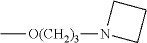 | —Cl | —H | —H | —H | —H |
| 23 | —H | —O(CH₂)₃—NH—CH₃ | —Cl | —H | —H | —H | —H |
| 24 | —H | 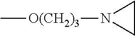 | —Cl | —H | —H | —H | —H |
| 25 | —H | —O(CH₂)₃—Cl | —Cl | —H | —H | —H | —H |
| 26 | —OCH₃ | —OCH₃ | —H | —H | —H | —H | —H |
| 27 | —OCH₃ | —OCH₃ | —H | —OCH₃ | —H | —H | —H |
| 28 | —OCH₃ | —OCH₃ | —OCH₃ | —H | —H | —H | —H |
| 29 | —OCH₃ | —OCH₃ | —CH₃ | —H | —H | —H | —H |
| 30 | —OCH₃ | —OCH₃ | —CF₃ | —H | —H | —H | —H |
| 31 | —OCH₃ | —OCH₃ | —F | —H | —H | —H | —H |
| 32 | —OCH₃ | —OCH₃ | —Cl | —H | —H | —H | —H |
| 33 | —OCH₃ | —OCH₃ | —Br | —H | —H | —H | —H |
| 34 | —OCH₃ | —OCH₃ | —F | —H | —H | —F | —H |
| 35 | —OCH₃ | —OCH₃ | —Cl | —H | —H | —F | —H |
| 36 | —OCH₃ | —OCH₃ | —OCH₃ | —H | —F | —H | —H |
| 37 | —OCH₃ | —OCH₃ | —OCH₃ | —H | —H | —F | —H |
| 38 | —OCH₃ | —OCH₃ | —OCH₃ | —H | —H | —H | —F |
| 39 | —OCH₃ | —OCH₃ | —OCH₃ | —H | —H | —F | —F |
| 40 | —OCH₃ | —OCH₃ | —OCH₃ | —H | —OH | —OCH₃ | —H |
| 41 | —OCH₃ | —OCH₃ | —OCH₃ | —H | —OCH₃ | —H | —H |
| 42 | —OCH₃ | —OCH₃ | —OCH₃ | —H | —H | —H | —OCH₃ |
| 43 | —OCH₃ | —OCH₃ | —OCH₃ | —H | —H | —CF₃ | —H |
| 44 | —OCH₃ | —OCH₃ | —OCH₃ | —H | —H | —H | —CF₃ |
| 45 | —OCH₃ | —OCH₃ | —OCH₃ | —H | —Cl | —H | —Cl |
| 46 | —OCH₃ | —OCH₃ | —OCH₃ | —H | —H | —Cl | —Cl |
| 47 | —OCH₃ | —OCH₃ | —OCH₃ | —H | —Br | —H | —H |
| 48 | —OCH₃ | —OCH₃ | —OCH₃ | —H | —NH₂ | —H | —H |
| 49 | —OCH₃ | —OCH₃ | —OCH₃ | —OCH₃ | —H | —F | —H |
| 50 | —OCH₂CH₃ | —OCH₂CH₃ | —OCH₃ | —H | —H | —H | —H |
| 51 | —OCH₂CH₃ | —OCH₂CH₃ | —Cl | —H | —H | —H | —H |
| 52 | —OCH₂CH₃ | —OCH₂CH₃ | —Br | —H | —H | —H | —H |
| 53 | —OCH₂—CH₂CH₃ | —OCH₂—CH₂CH₃ | —Cl | —H | —H | —H | —H |
| 54 | —OCH₂Ph | —OCH₃ | —OCH₃ | —H | —H | —H | —H |
| 55 | —O(CH₂)₂—O—CH₃ | —O(CH₂)₂—O—CH₃ | —Br | —H | —H | —H | —H |
| 56 | —O(CH₂)₂—N—(CH₃)₂ | —O(CH₂)₂—N—(CH₃)₂ | —Cl | —H | —H | —H | —H |
| 57 | —O(CH₂)₂—N—(CH₃)₂ | —O(CH₂)₂—N—(CH₃)₂ | —Br | —H | —H | —H | —H |
| 58 | —O(CH₂)₂—N—CH₂CH₃ | —O(CH₂)₂—NH—CH₂CH₃ | —Cl | —H | —H | —H | —H |
| 59 | 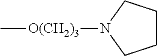 | 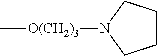 | —Cl | —H | —H | —H | —H |

Particularly preferred compounds of the invention are according to general formula (Ig)

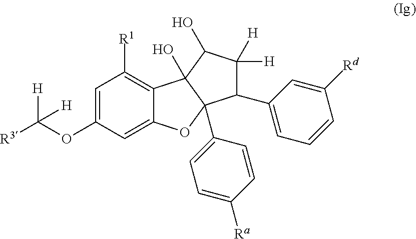

wherein
R¹ is selected from —H and —OCH₃;
R³′ is selected from —H; —CH₂NHCH₃; —CH₂N(CH₃)₂; and —CH₂— substituted with

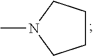

### R^{a} is —F, —Cl or —OCH₃; and

### R^{d}—H, —F or —Cl.

Particularly preferred compounds of one of the general formulae (I), (Ia), (Ib), (Ic), (Id), (Ie), (If) or (Ig) are selected from the group consisting of

(1,3,3a,8b)-3-(3-fluorophenyl)-6,8-dimethoxy-3a-(4-methoxyphenyl)-2,3,3a,8b-tetrahydro-1H-benzo[d]cyclopenta[b]furan-1,8b-diol (for the purpose of the specification also referred to as “IMD-019064”):

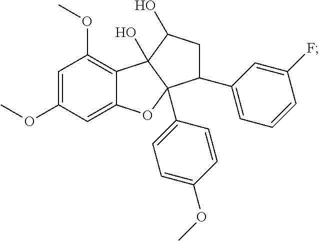

(1,3a,8b)-3a-(4-chlorophenyl)-3-phenyl-6-(2-(pyrrolidin-1-yl)ethoxy)-2,3,3a,8b-tetrahydro-1H-benzo[d]cyclopenta[b]furan-1,8b-diol (for the purpose of the specification also referred to as “IMD-026259”; cf. WO 2005/113529, example 33):

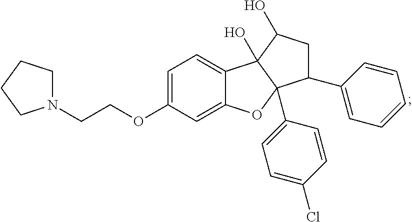

and

(1,3a,8b)-3a-(4-chlorophenyl)-6-(2-(methylamino)ethoxy)-3-phenyl-2,3,3a,8b-tetrahydro-1H-benzo[d]cyclopenta[b]furan-1,8b-diol (for the purpose of the specification also referred to as “IMD-026260”; cf. WO 2005/113529, example 34):

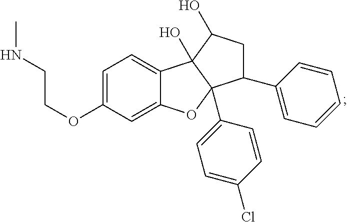

and the physiologically acceptable salts thereof.

A further aspect of the invention relates to the use of at least one compound of general formula (I), preferably of general formula (1a), (1b), (1c), (1e), (1f) or (1g), according to the invention for the manufacture of a medicament for the treatment and/or prophylaxis of angiogenesis-related disorders, preferably pulmonary hypertension. All preferred embodiments of the compound of general formula (I) also apply to the medicament according to the invention and thus, are not repeated here below.

Preferably, the medicament is a pharmaceutical composition comprising at least one compound of general formula (I) according to the invention as described above and a physiologically acceptable carrier.

A further aspect of the invention relates to the use of at least one compound of general formula (I) according to the invention for the manufacture of a pharmaceutical composition as described above for the treatment and/or prophylaxis of angiogenesis-related disorders preferably pulmonary hypertension. All preferred embodiments of the compound of general formula (I) also apply to the pharmaceutical composition according to the invention and thus, are not repeated here below.

The pharmaceutical composition according to the invention may be liquid, e.g. a solution, dispersion, suspension or emulsion; or solid, e.g. a powder, paste, gel, and the like.

Suitable physiologically acceptable carriers are known to the person skilled in the art. Suitable liquid carriers include water, ethanol and the like. Suitable solid carriers include typical pharmaceutical excipients, such as fillers, binders, glidants, disintegrants, and the like. In this regard it can be referred to, e.g., D. E. Bugay et al., Pharmaceutical Excipients, Informa Healthcare; 1 edition (Dec. 1, 1998). In general, the pharmaceutical composition according to the invention may contain inert non-toxic pharmaceutically suitable auxiliaries, such as for example excipients, solvents, vehicles, emulsifiers and/or dispersants.

The following auxiliaries can be mentioned as examples: water, solid excipients such as ground natural or synthetic minerals (e.g. talcum or silicates), sugar (e.g. lactose), non-toxic organic solvents such as paraffins, vegetable oils (e.g. sesame oil), alcohols (e.g. ethanol, glycerol), glycols (e.g. polyethylene glycol), emulsifying agents, dispersants (e.g. polyvinylpyrrolidone) and lubricants (e.g. magnesium sulphate).

The relative weight ratio of the compound of general formula (I) and the physiologically acceptable carrier is preferably within the ratio of from 99.9:0.1 to 0.1:99.9.

A further aspect of the invention relates to the use of at least one compound of general formula (I) according to the invention for the manufacture of a pharmaceutical dosage form containing the pharmaceutical composition as described above for the treatment and/or prophylaxis of angiogenesis-related disorders. All preferred embodiments of the compound of general formula (I) also apply to the pharmaceutical dosage form according to the invention and thus, are not repeated here below.

The pharmaceutical dosage forms according to the invention may be adapted, e.g., for systemic, local or topical administration. Systemic administration includes, e.g., intravenous, inhalative or oral administration.

The compounds according to the invention can exhibit non-systemic or systemic activity, wherein the latter is preferred. To obtain systemic activity the pharmaceutical dosage forms containing the active compounds can be administered, among other things, orally, parenterally, or inhalatory, wherein oral administration is preferred. To obtain non-systemic activity the pharmaceutical dosage forms containing the active compounds can be administered, among other things, topically.

For parenteral administration, pharmaceutical dosage forms for administration to the mucous membranes (i.e. buccal, lingual, sublingual, rectal, nasal, pulmonary, conjunctival or intravaginal) or into the interior of the body are particularly suitable. Administration can be carried out by avoiding absorption (i.e. intracardiac, intra-arterial, intravenous, intraspinal or intralumbar administration) or by including absorption (i.e. intracutaneous, subcutaneous, percutaneous, intramuscular or intraperitoneal administration).

For the above purpose the pharmaceutical dosage forms containing the active compounds can be administered per se or in pharmaceutical dosage forms (administration forms).

Suitable pharmaceutical dosage forms for oral administration are, inter alia, normal and enteric-coated tablets, capsules, coated tablets, pills, granules, pellets, powders, solid and liquid aerosols, syrups, emulsions, suspensions and solutions. Suitable pharmaceutical dosage forms for parenteral administration are injection and infusion solutions.

The active compound can be present in the pharmaceutical dosage forms in concentrations of from 0.001-100% by weight; preferably the concentration of the active compound should be 0.5-90% by weight, i.e. quantities which are sufficient to allow the specified range of dosage.

In the case of oral administration, tablets can of course also contain additives such as sodium citrate as well as additives such as starch, gelatin and the like. Flavour enhancers or colorants can also be added to aqueous preparations for oral administration.

For the obtainment of effective results in the case of parenteral administration it has generally proven advantageous to administer quantities of about 0.0001 to 100 mg/kg, preferably about 0.001 to 10 mg/kg, more preferable about 0.01 to 1 mg/kg of body weight. In the case of oral administration the quantity is about 0.001 to 100 mg/kg, preferably about 0.1 to 50 mg/kg of body weight.

In spite of this, it can be necessary in certain circumstances to depart from the amounts mentioned, namely as a function of body weight, application route, individual behaviour towards the active component; manner of preparation and time or interval at which application takes place. It can for instance be sufficient in some cases to use less than the aforementioned minimum amount, while in other cases the upper limit mentioned will have to be exceeded. In the case of the application of larger amounts, it can be advisable to divide them into a plurality of individual doses spread through the day.

The percentages in the tests and examples which follow are, unless otherwise stated, by weight; parts are by weight. Solvent ratios, dilution ratios and concentrations reported for liquid/liquid solutions are each based on the volume.

The pharmaceutical dosage forms may exhibit, e.g., an immediate or a sustained release profile of the active compound contained therein.

The compounds of the invention are inhibitors of HIF-1 protein accumulation, and can therefore be used for the manufacture of a medicament intended to inhibit HIF-1 protein accumulation.

The compounds according to the invention exhibit an unforeseeable, useful pharmacological and pharmacokinetic activity spectrum. They are therefore suitable for use as medicaments for the treatment and/or prophylaxis of disorders in humans and animals.

The compounds of the general formula (I) are HIF inhibitors and therefore suitable for the treatment and/or prophylaxis of a variety of angiogenesis-related disorders or are useful to prepare a medicament for the treatment and/or prophylaxis of angiogenesis-related disorders.

In general, the compounds of general formula (I) according to the invention can be used for the treatment and/or prophylaxis of angiogenesis-related disorders or are useful to prepare a medicament for the treatment and/or prophylaxis of angiogenesis-related disorders.

Angiogenesis-related disorders are preferably selected from the group consisting of diseases of the urogenital tract, preferably non-inflammatory diseases of the female genital tract, endometriosis of the uterus, endometriosis of the ovary, endometriosis of tuba uterine, endometriosis of the intestine, endometriosis of scars, endometriosis of septum recto-vaginale, endometriosis of the vagina, and endometriosis of the pelvis peritoneum; eye diseases, preferably macular degeneration, vitelliform dystrophy (Best disease), retino-pathies, diabetic retinopathy, glaucoma, neuroscular glaucoma, choroidal neovascularisation, occult choroidal neovascularisation, neovascularisation of the cornea, retrolental fibroplasias, and rubeosis irridis; lung diseases, preferably airway remodelling, COPD (chronic obstructive respiratory disorder), ARDS (acute respiratory distress syndrome), infant respiratory distress syndrome, pulmonary hypertension, pulmonary sarcoidosis, and idiopathic pulmonary fibrosis; kidney diseases, preferably nephropathies, chronic hypoxia induced diseases, ESRD, renal fibrosis, renal artery stenosis, and glomerulonephritis; osteoarthritis; preferably gonarthrosis, coxarthrosis, polyarthrosis, rhizarthrosis, and further athroses; rheumatic disorders, preferably rheumatoide arthritis; bone diseases, preferably osteoporosis and chondrocyte related disorders; myocardial angiogenesis; metastasis; endometriosis; wound healing; erectile dysfunction, preferably Peyronies disease; benign proliferative diseases, preferably benign tumors; hemangiomas, preferably liver hemangioma, cavernous hemangioma, and Klippel-Trenaunay-Weber (KTW) syndrome; skin disorders, preferably scleroderma; anemia, preferably erythropoesis; systemic diseases, preferably systemic sclerosis, and sarcoidosis; resistance reducer, preferably radiosensitisation, chemosensitisation, and drug resistance reducer; pediatric malignancies; tissue engineering; apoptosis stimulation.

In a particularly preferred embodiment of the invention, the compounds are for treatment or prevention of pulmonary disorders selected from the group consisting of pulmonary hypertension, pulmonary sarcoidosis, and idiopathic pulmonary fibrosis. Such diseases are generally regarded as angiogenesis-related. Nonetheless, for the purpose of the specification, the terms “pulmonary hypertension”, “pulmonary sarcoidosis”, and “idiopathic pulmonary fibrosis” refers to any pulmonary hypertension, pulmonary sarcoidosis, and idiopathic pulmonary fibrosis, respectively, irrespective of whether they are angiogenesis-related or not, if any.

In a preferred embodiment of the invention, angiogenesis-related disorders are selected from the group consisting of diseases of the urogenital tract, preferably non-inflammatory diseases of the female genital tract, endometriosis of the uterus, endometriosis of the ovary, endometriosis of tuba uterine, endometriosis of the intestine, endometriosis of scars, endometriosis of septum rectovaginale, endometriosis of the vagina, and endometriosis of the peritoneum.

In another preferred embodiment of the invention angiogenesis-related disorders are selected from the group consisting of eye diseases, preferably selected from the group consisting of macular degeneration, vitelliform dystrophy (Best disease), retinopathies, diabetic retinopathy, glaucoma, neuroscular glaucoma, choroidal neovascularisation, occult choroidal neovascularisation, neovascularisation of the cornea, retrolental fibroplasias and rubeosis irridis.

In another preferred embodiment of the invention angiogenesis-related disorders are selected from the group consisting of lung diseases, preferably selected from the group consisting of airway remodelling, COPD (chronic obstructive respiratory disorder), ARDS (acute respiratory distress syndrome), infant respiratory distress syndrome, pulmonary hypertension, pulmonary sarcoidosis and idiopathic pulmonary fibrosis.

In another preferred embodiment of the invention angiogenesis-related disorders are selected from the group consisting of kidney diseases, preferably nephropathies, chronic hypoxia induced diseases, ESRD, renal fibrosis, renal artery stenosis, and glomerulonephritis.

In another preferred embodiment of the invention, angiogenesis-related disorders are selected from the group consisting of osteoarthritis, preferably gonarthrosis, coxarthrosis, polyarthrosis, rhizarthrosis, and further arthroses.

In another preferred embodiment of the invention, angiogenesis-related disorders are selected from the group consisting of rheumatic disorders, preferably rheumatoide arthritis.

The compounds according to general formula (I) of the invention can be synthesized by various routes. For example, the compounds can be prepared fully synthetically, starting from building blocks that are commercially available. Furthermore, the compounds according can be isolated from plants, preferably from various species of the Aglaia plant or the precursor products isolated from said plants can be used as starting materials in the synthesis (semi-synthetic route). Thus, compounds of general formula (I) can be obtained by isolation, by semi-synthetic derivatization of the compounds obtained by isolation or by synthesis following previously published or new synthetic route. In other words, the compounds according to the invention can be, e.g., natural products, derivatives of these natural products or total synthetic analogs.

For the purpose of the specification, “HIF-1” does describe the protein while “Hif-1” describes the gene/mRNA.

Some preferred methods for the preparation of compounds of general formula (I) are described here below:

### EXAMPLES

### Example 1

### Suppression of Hypoxia Induced HIF-1 Dependent Luciferase Expression

### Dose-Response Curve of the Compounds on a HIF-1 Luciferase-Dependent Reporter Gene Activity in 2 Cell Lines (Jurkat T and 293T Cells)

A HIF-1-dependent luciferase reporter gene was transfected into 293T cells by transfection with Rotifect™ (Trademarked by Carl Roth GmbH; Karlsruhe, Germany) or alternatively into Jurkat T cells by electroporation. Both cell lines were transfected with a HIF-1-dependent reporter gene. The next day, cells were preincubated for 1 h with the indicated, submicromolar concentrations of the test compounds and then incubated for further 8 h under normoxic or hypoxic (1% O₂) conditions. After that, cells were harvested followed by the analysis of luciferase activity in a luminometer.

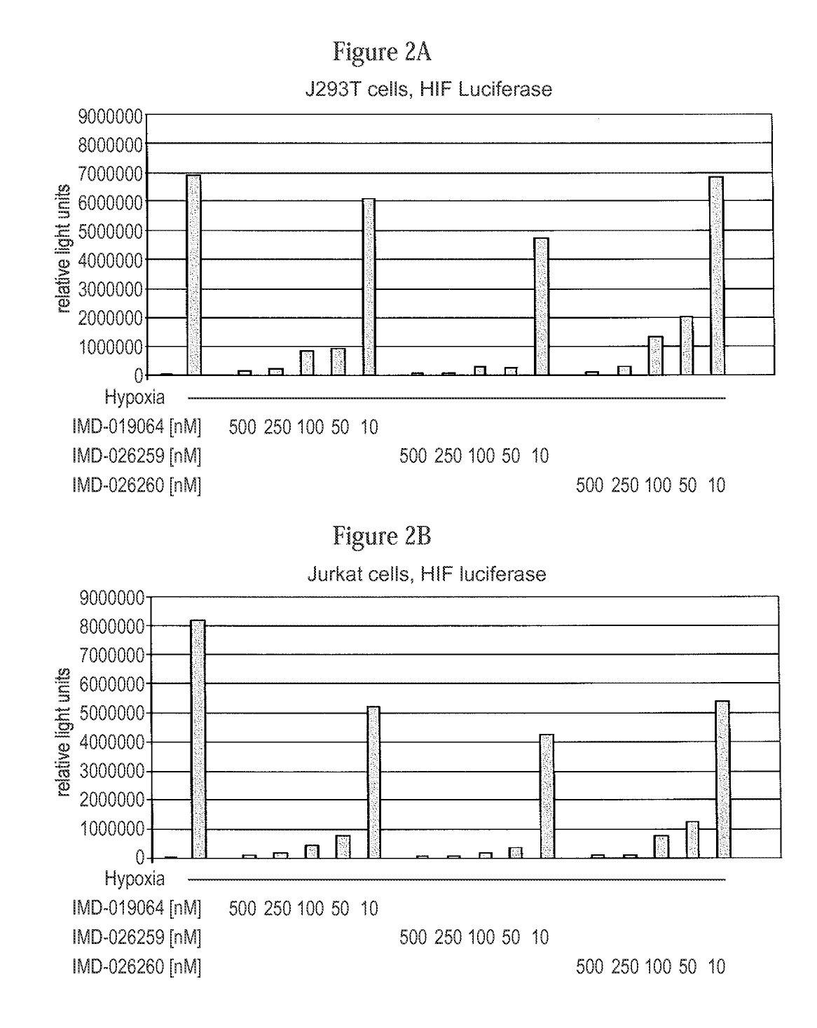

The results for 3 exemplary compounds are shown in FIGS. 2a (293T cells) and 2b (Jurkat T cells). Relative light units are shown, the experiments were performed in triplicates, mean values are given.

Analysis of luciferase activity in a luminometer revealed significant inhibition of HIF-1 mediated luciferase activity in 239T cells (FIG. 2a) and Jurkat cells (FIG. 2b) at compound concentrations of 50 nM of each compound significantly blocked HIF-1 activity, while 10 nM showed only a minor impact on HIF-1-dependent transcription.

### Example 2

### IC₅₀ Value Determination of Compounds with HIF-1 Luciferase Inhibitory Activity

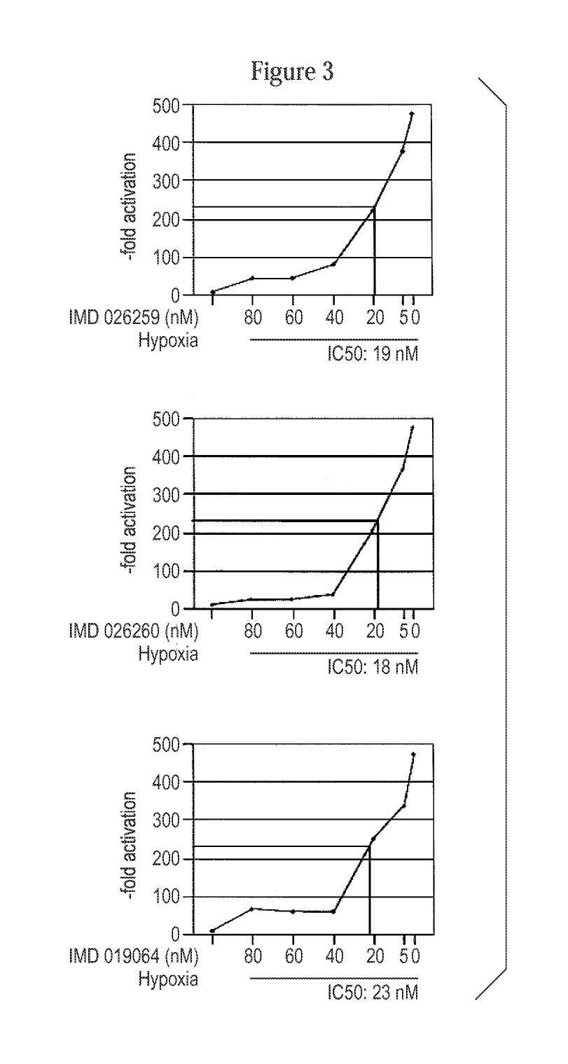

A HIF-1-dependent luciferase reporter gene was transfected into 293T cells. The next morning, cells were pretreated for 1 h with the indicated concentrations of compounds, followed by induction of hypoxia (hypoxia chamber) for 8 h. Subsequently cells were harvested and lyzed. Luciferase activity in cell extracts was measured in a luminometer (Duo Lumat LB 9507, Berthold) by injecting 20 microliter of assay buffer to 20 microliter of extract. Light emission was measured for 10 s, relative light units are given. The results are presented in FIG. 3 and show the IC₅₀ values of 19, 18, and 23 nM which were found for IMD-026259, IMD-026260 and IMD-019064, respectively.

### Example 3

### Time-Dependency of Compound Intervention on HRE-Luciferase Expression

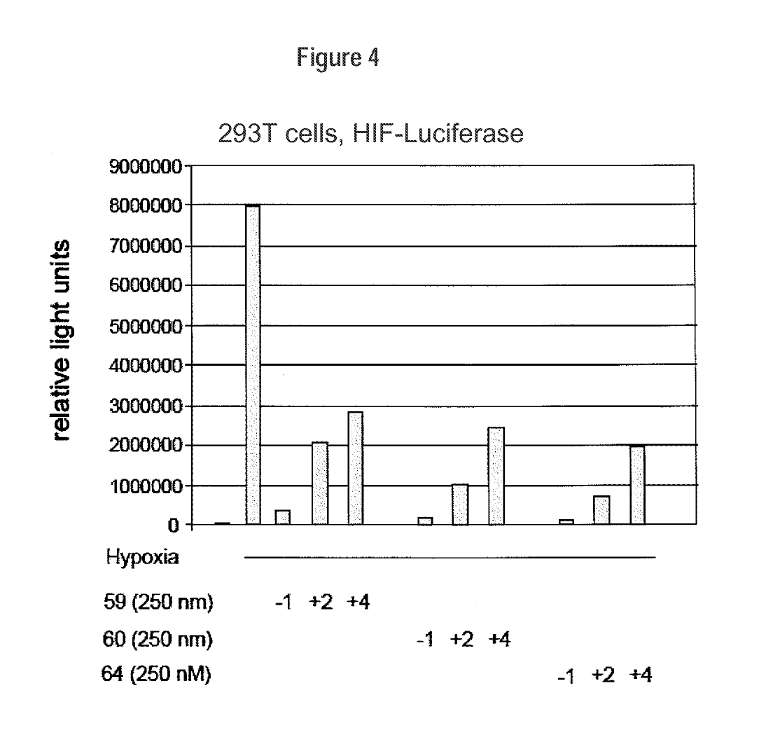

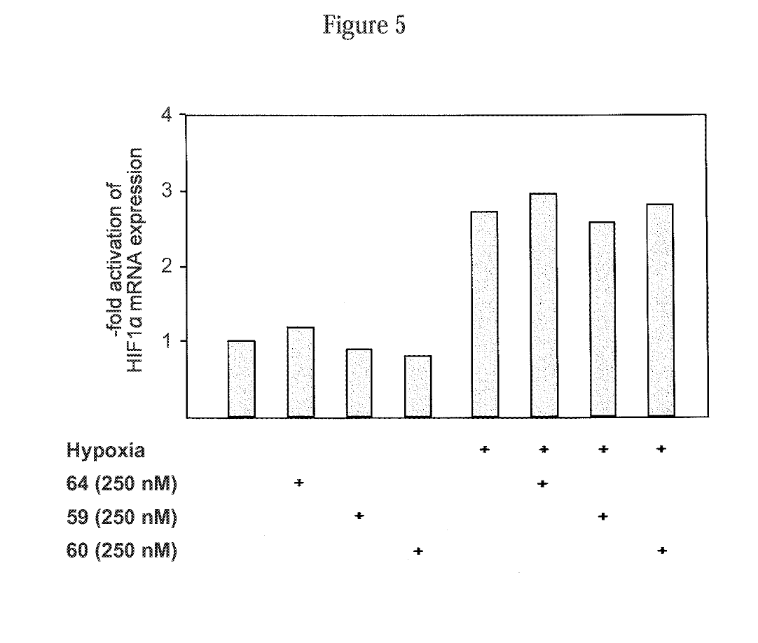

After transfection of 293T cells with a HIF-1-dependent luciferase reporter gene, cells were further incubated under normoxic or hypoxic (8 h, 1% O₂) conditions as shown in FIG. 4. Effectively blocking concentrations of the IMDs (250 nM each) were added either 1 hour prior to hypoxia or 2 and 4 hours after induction of hypoxia.

IMD-019064; IMD-026259 and IMD-026260 were shown to be able to inhibit HIF-1-dependent luciferase expression even when added 4 hours after hypoxic induction. These results demonstrate that the compounds of the invention do not only prevent the induction of the HIF-1 response but also interfere with ongoing HIF-1-dependent transcription when the protein is already stabilized (FIG. 4).

### Example 4

### Effect of Compounds on Hif-1 mRNA Expression

In order to investigate whether the compounds of the invention do exert their HIF-1-1 suppressive action via inhibition of HIF-1-1 transcription the compounds were tested for their effects on HIF-1α mRNA production. In all experiments, human cells were exposed to efficiently blocking concentrations (250 nM) of the test compounds for 3 h. Then cells were further cultivated under normoxic or hypoxic conditions (45 minutes, 1% O₂) respectively. Afterwards cell samples were harvested and RNA was extracted. cDNA was synthesized using specific primers and the PE Applied Biosystems Reverse Transcription Reagents. Real-time PCR was performed by using the SYBR Green I detection chemistry (Applied Biosystems) and an ABI Prism 7300 system. The expression of Hif-1α was quantified and results were normalized to Hprt1 and β-Actin (“housekeeping-gene”) expression. The relative abundance of the different genes was calculated by the comparative CT method. HIF-1α transcription of normoxic control cells was arbitrarily set to 1. A representative experiment is displayed in FIG. 5. While hypoxia moderately increased HIF-1α transcription by a factor of ˜3 under the conditions employed, the quantification of HIF-1α mRNA expression by qPCR revealed no significant effect of the tested compounds. The tested substances (IMD-019064; IMD-026259 and IMD-026260) did not show any effects on Hif-1α mRNA production—neither under normoxic nor under hypoxic conditions. Therefore an effect of IMD-compounds on HIF-1-dependent gene expression seems not to be mediated on HIF-1α transcription (FIG. 5).

### Example 5

### Effect of IMD Compounds on HIF-1 Target Gene Expression

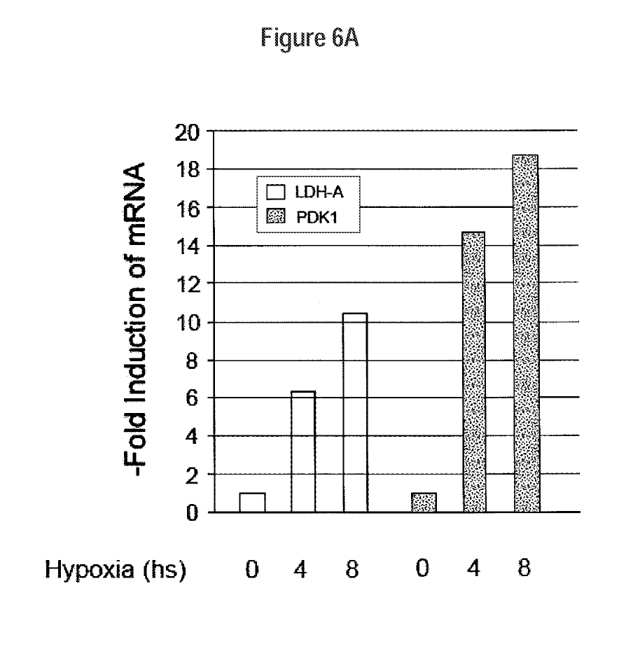

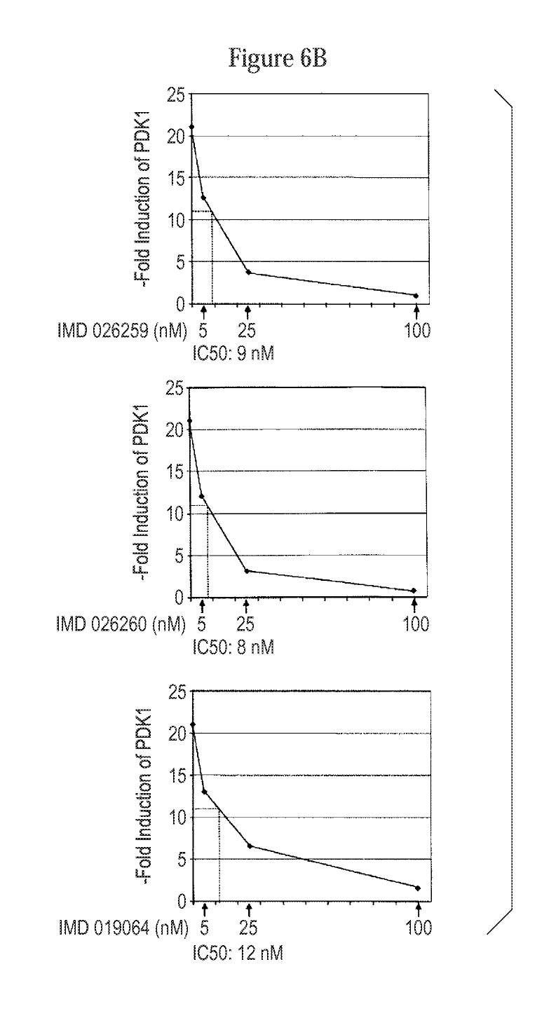

Human 293T cells were cultivated either under normoxic conditions or under hypoxic conditions (1% oxygen) for 4 and 8 hs respectively. Cells were harvested, followed by the isolation of RNA and the generation of cDNAs from Oligo (dT)20 primers using a Reverse Transcriptase kit. Then gene expression was measured by real-time PCR for the following two selected HIF-1α target genes: LDH-A (lactate dehydrogenase isoform A), and PDK1 (pyruvate dehydrogenase kinase1). Real-time PCR was performed with cDNA using specific primers and the SYBR Green I detection chemistry system (Applied Biosystems), utilizing an ABI Prism 7300 system. Actin was also measured as an internal control. The relative abundance of the different genes was calculated by the comparative CT method. In order to facilitate comparison, gene expression under normoxic conditions was arbitrarily set to 1 for each of the three genes. Two independent experiments confirmed PDK1 as the gene which was induced most prominently compared to LDH. The results are shown in FIG. 6a.

To investigate the capability of the compounds to inhibit PDK1, 293T cells were pretreated for 1 h with 3 different concentrations of the respective inhibitors (IMD-019064; IMD-026259; and IMD-026260). Subsequently, cells were incubated under normoxic/hypoxic conditions for further 8 h and the expression of PDK1 was quantified by real-time PCR as described above. The results are displayed in FIG. 6b and show that all three inhibitors potently suppress the activation of the endogenous PDK1 gene. The IC50 values were determined to range from 8 to 12 nM (IMD 026259, 9 nM; IMD 026260, 8 nM; and IMD 019064, 12 nM), indicating that endogenous genes (e.g. PDK1) are even more potently suppressed than the reporter genes (e.g. luciferase).

Furthermore, the capability of IMD-019064, IMD-026259 and IMD-026260 to specifically repress HIF-1α-dependent target gene expression was demonstrated.

### Example 6

### Suppression of Hypoxia Induced HIF-1α Protein Accumulation

### Determination of the Dose-Dependence of Compounds for the Prevention of Hypoxia-Induced HIF-1 Stabilisation

Effects of IMD-026259, IMD-026260 and IMD-019064 on the stabilisation/accumulation of HIF-1α protein were investigated. 293T cells were preincubated with the indicated concentrations of all three compounds. After 1 h of pre-incubation, 293T cells were pre-incubated with the indicated concentrations of compounds and then further incubated under normoxic or hypoxic (4 h, 1% O₂) conditions respectively. After lysis of cells in 1×SDS sample buffer and sonication, the samples were analyzed for HIF-1α abundance by immunoblotting. Equal amounts of protein contained in lysates were separated by reducing SDS-PAGE and then further analyzed by immunoblotting with antibodies specifically recognizing HIF-1α and the loading control Actin (demonstrating loading of equal protein amounts).

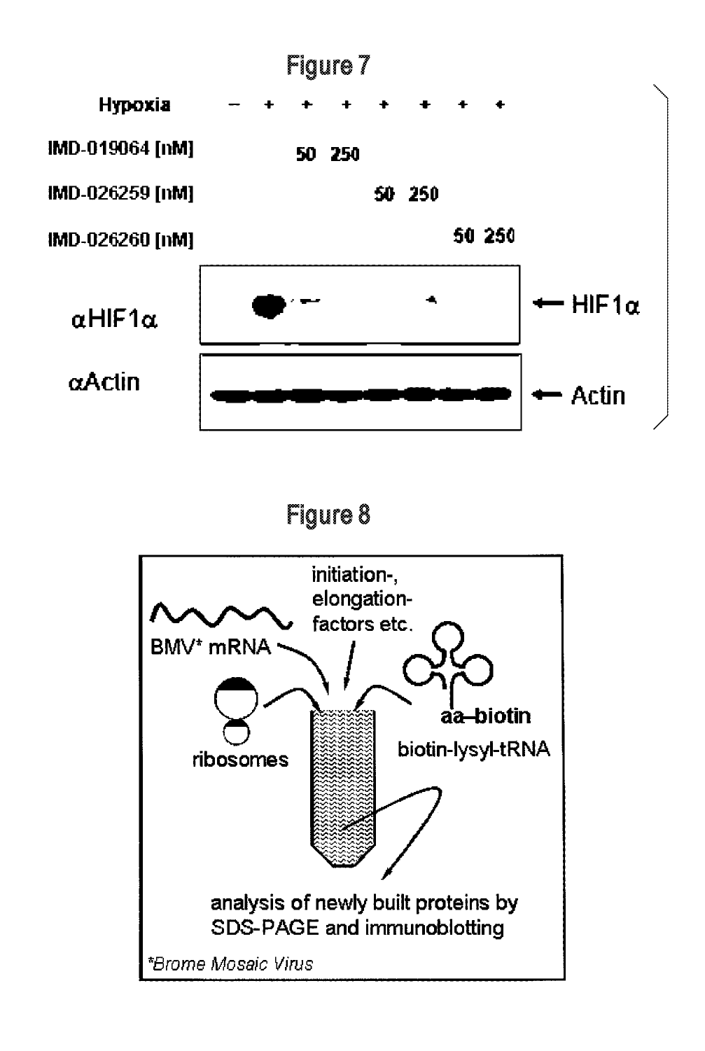

As depicted in FIG. 7, the accumulation of HIF-1α protein was not detectable at normoxic conditions (−) while being induced by hypoxia (+). The HIF-1α protein accumulation was significantly and specifically prevented at compound concentrations of 50 nM. The concentration of 250 nM of IMD-019064 and IMD-026260 did almost completely block HIF-1α protein accumulation.

### Example 7

### Inhibition of Cell-Free Protein Translation

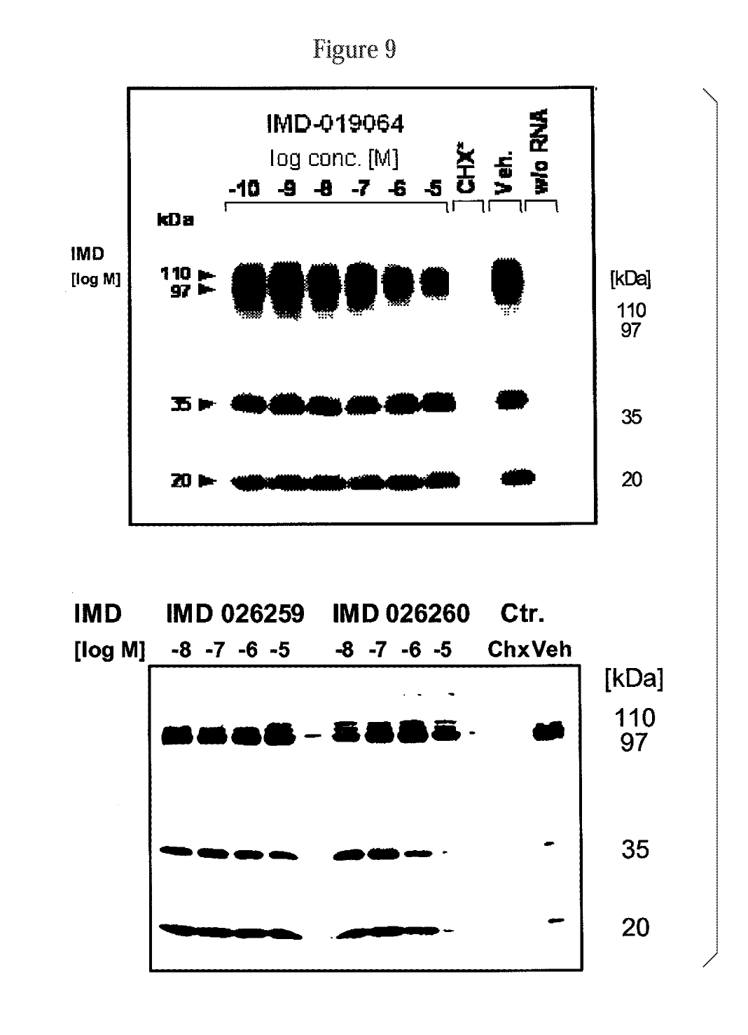

The effect of IMD-019064 on cell-free protein translation in vitro was investigated (see also assay principle in FIG. 8). Brome mosaic virus (BMV) mRNA encoding for 4 different viral proteins was incubated with ribosomes in the presence and absence of IMD-019064 (FIG. 9a), IMD-026259, IMD-026260 (FIG. 9b), or cycloheximide (Chx, 10 μM) (positive control) and the formed proteins analyzed by SDS-PAGE.

As shown in FIG. 9a, IMD-019064 did not affect protein synthesis in vitro whereas cycloheximide potently suppressed formation of newly synthesized proteins (FIG. 9a, lane: Chx). No significant effect of IMD-019064 (FIG. 9a), IMD-026259, IMD-026260 (FIG. 9b), on in vitro protein translation was observed at relevant (nM) concentrations while cycloheximide (Chx; positive control) did suppress protein translation. Panels show representative SDS-PAGE protein gels. Vehicle-containing translation reactions (Veh) represent 100% translation efficacy, while Cycloheximide (Chx; 10 μg/ml) a known inhibitor of protein translation that was used as a positive control does show significant protein reduction.

### Example 8

### Inhibition of Cell-Free Protein Translation

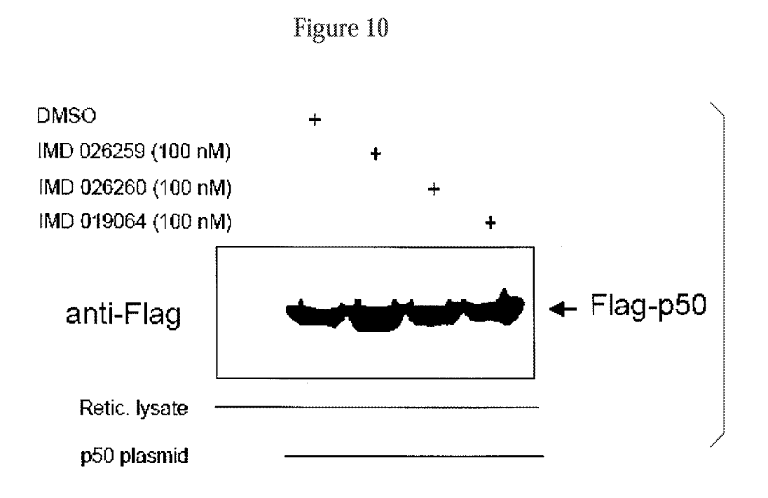

Effects of the compounds on in vitro translation. The TNT® Coupled Reticulocyte Lysate Systems (cell free protein expression) was used to test any potential effects on translation. Flag-tagged NF-κB p50 protein was produced in vitro. The reticulocyte system was used including 100 nM of the respective compounds (IMD-019064; IMD-026259 and IMD-026260) and DMSO as a solvent control. After non-radioactive in vitro translation of Flag-tagged NF-κB p50 protein, proteins were separated by SDS-PAGE and the produced p50 protein was detected by immunoblotting using Flag antibodies (see FIG. 10).

These results revealed no detectable effects of the compounds of the invention on cell-free in vitro translation and—together with results from Example 7—make direct and un-specific effects of tested compounds on protein synthesis/translation highly improbable.

### Example 9

### Specific Inhibition of Signal-Dependent Amino Acid Incorporation in Cells

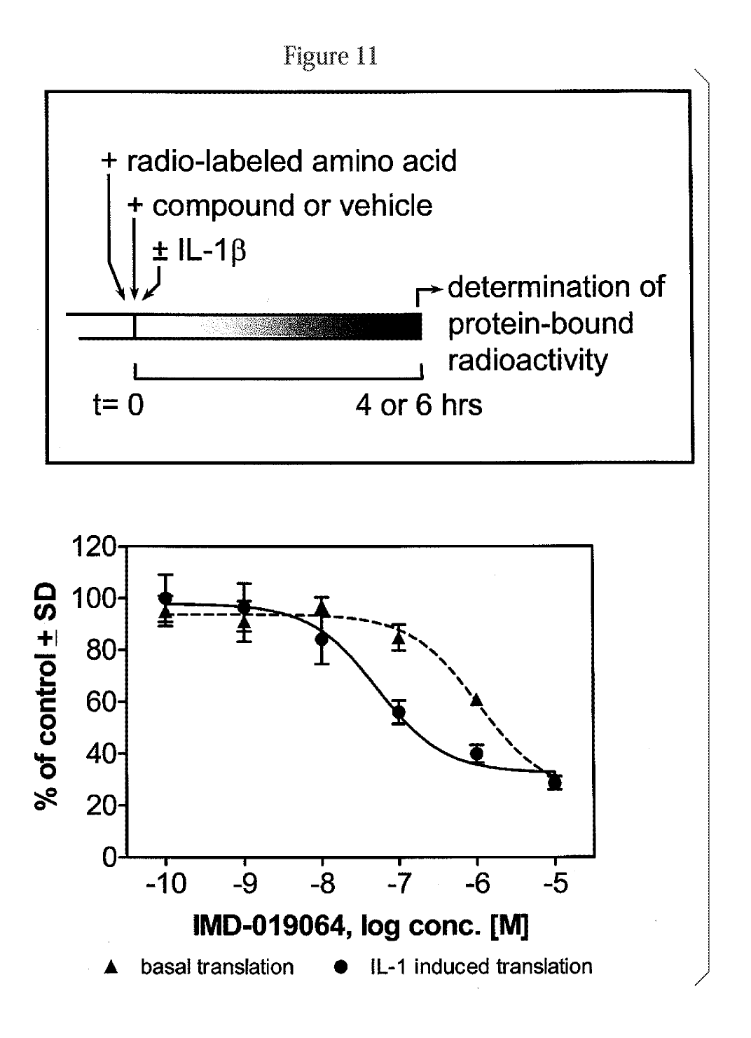

Compounds of the formula (I) have been reported in the literature to be un-specific inhibitors of protein synthesis. Thus, the question whether IMD-019064 (a rocaglaol derivative) is a direct inhibitor of protein synthesis has been addressed at different levels. Cellular assays (incorporation of radiolabeled amino acids into unstimulated and IL-1b-stimulated endothelial cells) were performed in order to investigate if inhibition of activated signaling cascades (e.g. IL-1 signalling pathway) may contribute to inhibitory activity of IMD-019064. In these studies a shift of the dose-response curve to the higher concentrations by a factor of 20 in un-stimulated cells compared to IL-1 stimulated cells was observed with respect to inhibition of amino acid incorporation. This indicates that inhibition of IL-1 dependent signaling indeed contributed to the protein synthesis-inhibitory activity of IMD-019064. IC₅₀ value of IMD-019064 action on cellular protein translation is significantly lowered after prior IL-1 induction. This cellular assay thereby indicates that inhibitory activity of IMD-019064 on cellular protein synthesis is mediated by specifically blocking e.g. IL-1 dependent signaling cascades (see FIG. 11).

### Example 10

### Inhibition of Cellular (In Vivo) Protein Translation

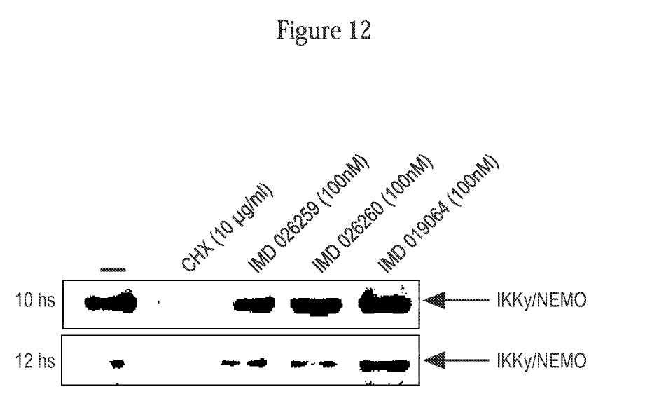

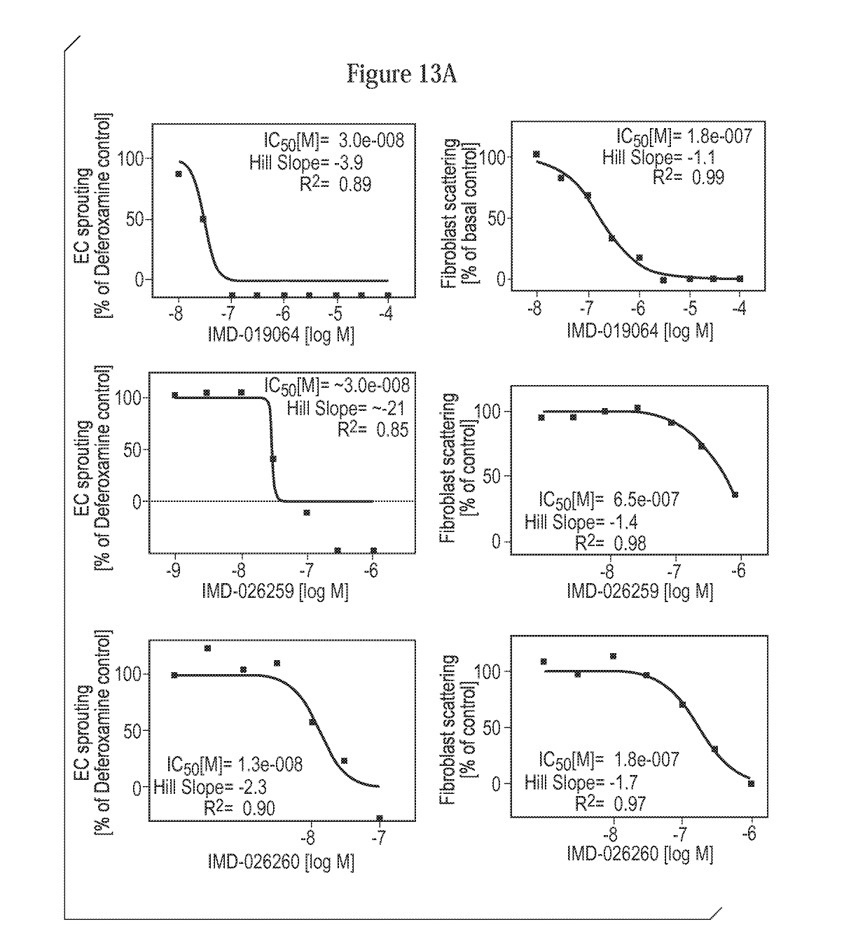

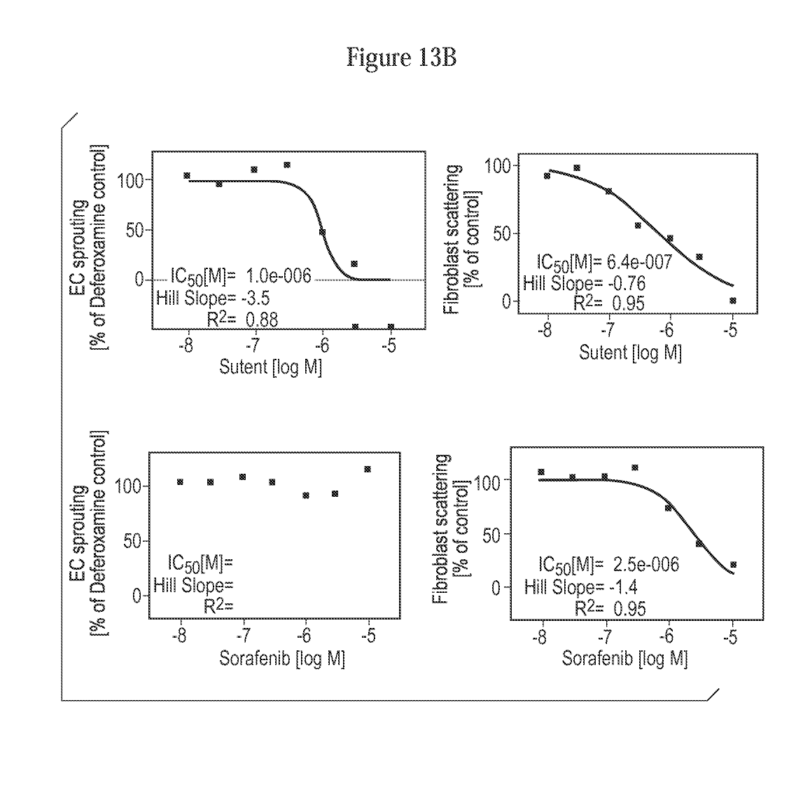

293T cells were grown in the presence of the known translation inhibitor cycloheximide (10 μg/ml) and 100 nM of IMD-019064, IMD-026259 and IMD-026260 respectively. The latter dose was chosen as it represents a concentration that is well above the IC50 values (approx. 5 to 10-fold) from gene expression studies (compare e.g. Examples 2 and 5) but also below concentrations that may cause non-specific effects. Cells were harvested after 10 h and 24 h and cell lysates were produced. Equal amounts of protein contained in the cell lysates were used for immunoblotting with anti-IKKy/NEMO antibodies as shown in FIG. 12. IKKy/NEMO is a constitutively expressed relatively labile protein. Thus successful inhibition of cellular protein translation should result in significant decline of anti-IKKy/NEMO signal. Treatment of 293T cells with the compounds IMD-019064, IMD-026259 and IMD-026260 did not result in reduced anti-IKKy/NEMO signals. These experiments confirmed the powerful inhibition of protein translation by cycloheximide, but did not reveal any un-specific inhibition of translation by any of the three compounds tested.

### Example 11

### Suppression of Angiogenic Sprouting in HUVEC Cells after Hypoxic Stimulus

Prior application the stock solution [100 mM] of IMD-019064 was diluted 1:10 in DMSO and serially diluted in half logarithmic steps covering the concentration range from 10 mM to 1 μM using 100% DMSO. The final assay concentration ranged from 100 μM to 10 nM with a final DMSO concentration in the assay of 1%. The experiments were pursued in modification of the originally published protocol (Korff and Augustin: J Cell Sci 112: 3249-58, 1999). In brief, spheroids were prepared as described by Korff and Augustin: J Cell Biol 143: 1341-52, 1998) by pipetting 500 endothelial cells (EC) in a hanging drop on plastic dishes to allow overnight spheroid aggregation. 50 EC spheroids were then embedded in 0.9 ml of a 3D collagen matrix and pipetted into individual wells of a 24 well plate to allow polymerization. The test compound, for HUVEC in combination with Deferoxamine [100 μM; induction of chemically induced hypoxia], was added after 30 min by pipetting 100 μl of a 10-fold concentrated working dilution on top of the polymerized gel (see Table 2 for final compound concentrations):

TABLE 2
Final compound concentrations [M]

| HUVEC | Concentration [M] + Deferoxamine [100 μM] | | | | | | | | |
|---|---|---|---|---|---|---|---|---|---|
| IMD-019064 | 1 × 10⁻⁴ | 3 × 10⁻⁵ | 1 × 10⁻⁵ | 3 × 10⁻⁶ | 1 × 10⁻⁶ | 3 × 10⁻⁷ | 1 × 10⁻⁷ | 3 × 10⁻⁸ | 1 × 10⁻⁸ |
| NHDF | Concentration [M] | | | | | | | | |
| IMD-019064 | 1 × 10⁻⁴ | 3 × 10⁻⁵ | 1 × 10⁻⁵ | 3 × 10⁻⁶ | 1 × 10⁻⁶ | 3 × 10⁻⁷ | 1 × 10⁻⁷ | 3 × 10⁻⁸ | 1 × 10⁻⁸ |

Plates were incubated at 37° C. for 24 hours and fixed by adding 4% paraformaldehyde. The cumulative sprout length of 10 randomly selected spheroids per data point was analyzed and the relative inhibition by the test compound determined. Fitting of IC₅₀ curves and calculation of IC₅₀ values was performed with GraphPad Prism 5.01. Sprouting intensity of EC and fibroblast spheroids was quantitated by an image analysis system using an inverted microscope and the digital imaging software Analysis 3.2 (Soft imaging system, Munster, Germany). In parallel NHDF (fibroblast) spheroids were embedded in a 3D collagen gel and treated for 24 h with different concentrations of IMD-019064. The cumulative sprout length of 10 randomly selected spheroids per data point was analyzed according to the procedure used for the HUVEC experiment.

The test compound IMD-019064 inhibits human umbilical vein endothelial cell (EC) sprouting and fibroblast scattering stimulated by chemically [Deferoxamine; 100 μM] induced hypoxia in a dose-dependent manner in the spheroid-based angiogenesis assay using a collagen matrix. Deferoxamine induced sprouting of HUVEC spheroids was significantly inhibited by IMD-019064 treatment. An IC₅₀ value of 30 nM could be determined (FIG. 13). NHDF (fibroblast) spheroid sprouting was found to be inhibited by IMD-019064 by an IC₅₀ value of 180 nM (FIG. 13). The findings did show a difference in HUVEC and fibroblast sensitivity against IMD-019064 by factor six. This demonstrates that inhibition of hypoxia induced angiogenic effects (HUVEC sprouting) is independent from unspecific cytotoxicity.

The same assays were performed with IMD-026260; IMD-026259; Sutent® (Sunitinib) and Sorafenib (Nexavar®) (see Table 3 for final compound concentrations).

TABLE 3
Final compound concentrations [M] as used in hypoxia
(Deferoxamine)-induced HUVEC sprouting assays.

| HUVEC | Concentration [M] + Deferoxamine [100 μM] | | | | | | |
|---|---|---|---|---|---|---|---|
| IMD-026260 | 1 × 10⁻⁷ | 3 × 10⁻⁸ | 1 × 10^{^{−}}⁸ | 3 × 10⁻⁹ | 1 × 10⁻⁹ | 3 × 10⁻¹⁰ | 1 × 10⁻¹⁰ |
| NHDF | Concentration [M] | | | | | | |
| IMD-0026260 | 1 × 10⁻⁶ | 3 × 10⁻⁶ | 1 × 10⁻⁷ | 3 × 10⁻⁸ | 1 × 10^{^{−}}⁸ | 3 × 10⁻⁹ | 1 × 10⁻⁹ |
| HUVEC | Concentration [M] + Deferoxamine [100 μM] | | | | | | |
| IMD-026259 | 1 × 10⁻⁷ | 3 × 10⁻⁸ | 1 × 10^{^{−}}⁸ | 3 × 10⁻⁹ | 1 × 10⁻⁹ | 3 × 10⁻¹⁰ | 1 × 10⁻¹⁰ |
| NHDF | Concentration [M] | | | | | | |
| IMD-0026259 | 1 × 10⁻⁶ | 3 × 10⁻⁶ | 1 × 10⁻⁷ | 3 × 10⁻⁸ | 1 × 10^{^{−}}⁸ | 3 × 10⁻⁹ | 1 × 10⁻⁹ |
| HUVEC | Concentration [M] + Deferoxamine [100 μM] | | | | | | |
| Sutent | 1 × 10⁻⁵ | 3 × 10⁻⁵ | 1 × 10⁻⁶ | 3 × 10⁻⁶ | 1 × 10⁻⁷ | 3 × 10⁻⁸ | 1 × 10^{^{−}}⁸ |
| NHDF | Concentration [M] | | | | | | |
| Sutent | 1 × 10⁻⁵ | 3 × 10⁻⁵ | 1 × 10⁻⁶ | 3 × 10⁻⁶ | 1 × 10⁻⁷ | 3 × 10⁻⁸ | 1 × 10^{^{−}}⁸ |
| HUVEC | Concentration [M] + Deferoxamine [100 μM] | | | | | | |
| Sorafenib | 1 × 10⁻⁵ | 3 × 10⁻⁵ | 1 × 10⁻⁶ | 3 × 10⁻⁶ | 1 × 10⁻⁷ | 3 × 10⁻⁸ | 1 × 10^{^{−}}⁸ |
| NHDF | Concentration [M] | | | | | | |
| Sorafenib | 1 × 10⁻⁵ | 3 × 10⁻⁵ | 1 × 10⁻⁶ | 3 × 10⁻⁶ | 1 × 10⁻⁷ | 3 × 10⁻⁸ | 1 × 10^{^{−}}⁸ |

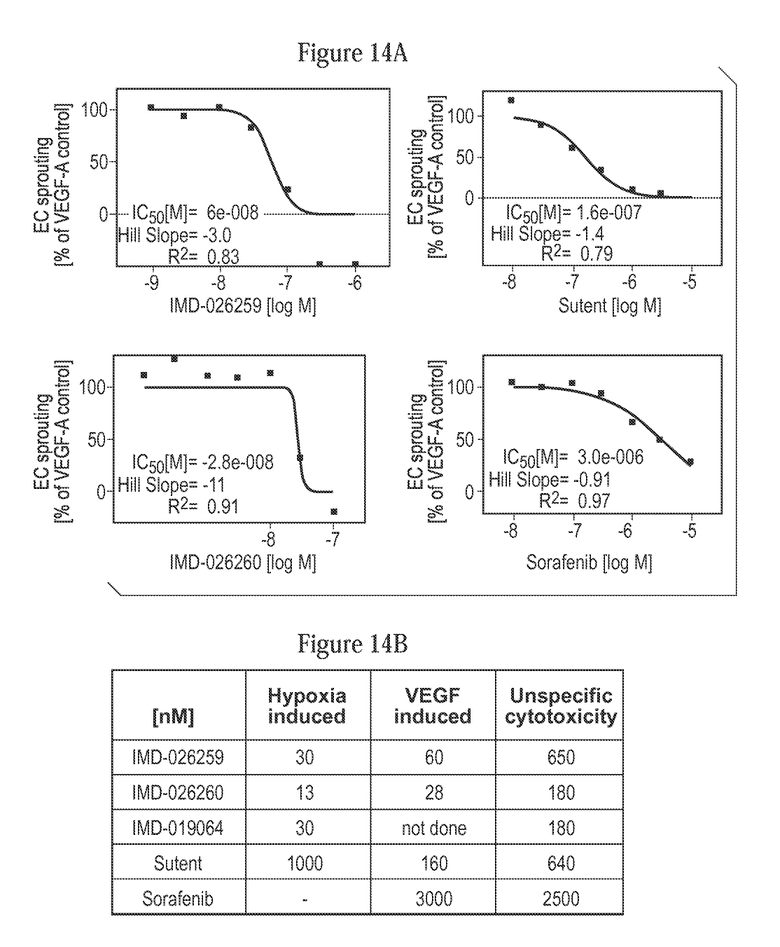

Results are presented in FIG. 13, respectively, indicating an IC₅₀ value of 13 nM (FIG. 13: Hypoxia induced EC sprouting) and an IC₅₀ value of 180 nM (FIG. 13: NHDF fibroblast scattering). These data (in comparison to IMD-019064) do show an even increased sensitivity of HUVECs resulting in a greater than thirteen-fold difference of HUVEC and fibroblast sensitivity against IMD-026260. This validates the IMD-019064 findings and demonstrates the improved inhibition of hypoxia induced angiogenic effects (HUVEC sprouting) independent from unspecific cytotoxicity by IMD-026260 (see FIG. 14b for summary of results).

### Example 12

### Suppression of Angiogenic Sprouting in HUVEC Cells after VEGF-A Stimulus

As described in Example 4 HUVEC spheroids were generated, cultivated and embedded in a 3D collagen gel and stimulated with Vascular Endothelial Growth Factor alpha [VEGF-A; 25 ng/ml] instead of deferoxamine. Cell spheroids were treated for 24 h with different concentrations of IMD-026259; IMD-026260; Sutent® (Sunitinib) and Sorafenib (Nexavar®) (in Molar [M], as indicated in Table 4; see also FIG. 14A).

TABLE 4
Final compound concentrations [M] as used in VEGF-induced HUVEC sprouting assays.

| HUVEC | Concentration [M] + VEGF-A [25 ng/ml] | | | | | | |
|---|---|---|---|---|---|---|---|
| IMD-026259 | 1 × 10⁻⁷ | 3 × 10⁻⁸ | 1 × 10⁻⁸ | 3 × 10⁻⁹ | 1 × 10⁻⁹ | 3 × 10⁻¹⁰ | 1 × 10⁻¹⁰ |
| HUVEC | Concentration [M] + VEGF-A [25 ng/ml] | | | | | | |
| IMD-026260 | 1 × 10⁻⁷ | 3 × 10⁻⁸ | 1 × 10⁻⁸ | 3 × 10⁻⁹ | 1 × 10⁻⁹ | 3 × 10⁻¹⁰ | 1 × 10⁻¹⁰ |
| HUVEC | Concentration [M] + VEGF-A [25 ng/ml] | | | | | | |
| Sutent | 1 × 10⁻⁵ | 3 × 10⁻⁵ | 1 × 10⁻⁶ | 3 × 10⁻⁶ | 1 × 10⁻⁷ | 3 × 10⁻⁸ | 1 × 10⁻⁸ |
| HUVEC | Concentration [M] + VEGF-A [25 ng/ml] | | | | | | |
| Sorafenib | 1 × 10⁻⁵ | 3 × 10⁻⁵ | 1 × 10⁻⁶ | 3 × 10⁻⁶ | 1 × 10⁻⁷ | 3 × 10⁻⁸ | 1 × 10^{^{−}}⁸ |

The cumulative sprout length of 10 randomly selected spheroids per data point was analyzed and the relative inhibition by the test compound determined. Fitting of IC₅₀ curves and calculation of IC₅₀ values was performed with GraphPad Prism® 5.01 (trademarked by GraphPad Software Inc.). VEGF-A induced sprouting of HUVEC spheroids was significantly inhibited by IMD-026260 treatment. An IC₅₀ value of 28 nM could be determined (FIG. 14a). Results from the examples described above are summarized in FIG. 14b IC50 values are given in nM for in vitro angiogenesis (HUVEC sprouting) studies after hypoxic or VEGF induction. By these data it is clearly shown that IMD-019064; IMd-026259 and IMD-026260 act as potent inhibitors of angiogenic sprouting in vitro. Furthermore it was demonstrated that the compounds of the invention have a potent inhibitory effect on HUVEC sprouting (neo-vascularisation) in vitro is superior to that of Sutent® (Sunitinib) and Sorafenib (Nexavar®).

### Example 13

### Evaluation of the Anti-Angiogenic Efficacy of Anti-Angiogenic Test Items in the Spheroid-Based In Vivo Angiogenesis Assay

Human endothelial cells (ECs; HUVECs) are applied subcutaneously in (n=10) SCID mice embedded in extracellular matrix components in the presence of smooth muscle cells (SMCs) and fibroblasts (NHDFs). The transplanted human ECs (EC spheroids in combination with ECs in suspension) form a complex and perfused three dimensional network of capillaries of human origin that is anastomosed (connected with) with the mouse vasculature and host (mouse) pericyte-covered. The quality of the newly formed vasculature is monitored by micro vessel density counting. Aim of the study is to investigate the anti-angiogenic potential of the test items in the spheroid-based in vivo angiogenesis assay. For this purpose HUVEC spheroids were prepared as described (Korff and Augustin: J Cell Biol 143: 1341-52, 1998) by pipetting 100 ECs in a hanging drop on plastic dishes to allow overnight spheroid formation. The following day EC spheroids were harvested and mixed in a Matrigel/fibrin solution with suspended HUVECs, SMCs and NHDFs. The final mixture to be used as a plug contained 100.000 spheroid ECs and 200.000 single suspended ECs, 300.000 SMCs and 100.000 NHDFs. SCID mice were subcutaneously injected with 500 μl of the cell/matrix suspension.

In the standard assay (with VEGF-A and FGF-2 stimulation and only ECs) the first perfused vessels are usually detected at day 4 to day 6. After 20 days of in vivo growth a well established vasculature with around 50-60% pericyte-covered and perfused vessels is usually observed. The SCID mice were treated with vehicle or with IMD-026260 (0.3 mg/ml; group 3) respectively, applied p.o. every 3^{rd} day.

The necropsy was conducted after all animals were weighed and anaesthetized. Afterwards mice were sacrificed by cervical dislocation and the Matrigel® (Trademark by BD Biosciences) plugs were removed. The Matrigel® plugs were photographed and fixed in 4% Roti-Histofix (Roth, Karlsruhe, Germany) at room temperature for 4-12 h. Thereafter the plugs were paraffin embedded using the semi-enclosed tissue processor Leica TP1020.

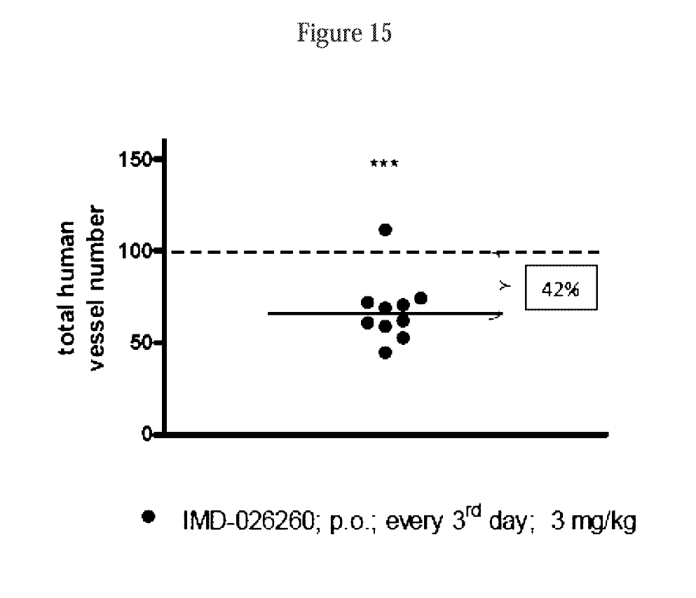

For histological examination of the human vasculature, paraffin sections (thickness=8-10 μm) were prepared from all plugs. Blood vessel formation was detected by staining the sections for human CD34 (NCL-END, Menarini, Berlin, Germany). Three sections per plug were analysed and three images were taken from each section at a magnification of 200× using the Eclipse TE2000-U microscope (Nikon, Kanagawa, Japan). The area analyzed per section (three images) was 0.44 mm². The vessel number (CD34 positive) was manually determined using the NIS-elements basic research software (Nikon, Kanagawa, Japan). Treatment with IMD-026260 resulted in a significant (pvalue=0.0001) 42% reduction of human vessel number compared to a vehicle control. The results of the study are graphically displayed as scatterplot (including the median bar) as indicated in FIG. 15.

### Example 14

### Reduction of Neurological Damage after Ischemic Injury (STROKE Model)

The method, which detects neuroprotective activity, follows that described by Longa E. Z., et al., (Stroke, 20, 84-91, 1989) and adapted by Esneault et al., (Neuroscience, 18; 152(2):308-20, 2008). Rats (Male Rj: Sprague-Dawley rats, weighing 250-350 g) are placed under isoflurane anaesthesia (5% for induction and 2% for maintenance, under 30% O2). Body temperature is monitored with a rectal temperature probe and maintained with a heating pad at 37° C.±1° C. throughout the experiment. Cerebral blood flow is continuously recorded by laser Doppler flowmetry (Moor Instruments MoorLAB) during a period covering induction of cerebral ischemia (from 10-15 minutes before and 5 minutes after MCAo). Under an operating microscope, a skin incision is made between the orbit and the ear and the temporal muscle is dissected. The laser Doppler probe is placed on the right lateral face of the skull.

After a midline incision of the neck, the right common carotid artery (CCA), the external carotid artery (ECA) and the internal carotid artery (ICA) are isolated from adjacent veins and nerves. The CCA is then ligatured and the ECA is electro-coagulated at 6±2 mm from its bifurcation from CCA. A nylon thread (0.18 mm diameter) with the extremity coated with translucent hot melt adhesive constitutes the embolus (3 mm length, 0.36-0.38 mm diameter). The embolus is inserted through a small incision into the ECA and is gently advanced into the ICA, until the cerebral blood flow decreases by 30-50% or a slight resistance is observed.

After intra-luminal ligature, the neck incision is sutured. The laser Doppler probe and the rectal temperature probe are removed. The rats recover from anaesthesia and are placed back in their home cages.

90 min later, rats are re-anaesthetized. The embolus and the ligature of the CCA are removed to allow reperfusion. The wounds are sutured and rats are placed back in their home cages. Rats receive an intraperitoneal (i.p.) administration of physiological saline (1 ml/day), during 5 days to prevent dehydration.

IMD-026259 will be evaluated at three doses (1, 10 and 100 μg/kg), i.v., 0, 2, 4 and 24 hours after reperfusion and compared to a vehicle control. The experiment will include a sham control group administered with the vehicle under the same experimental conditions. The experiment will therefore include 5 groups. 12 rats are studied per group. The test is performed blind.

### Neurological Score

The Neurological Score is assessed according to a modified version of the method of Bederson and al. (Stroke, 1986, 17(3): 472-476).

The test consists of 14 subsets as described below (table 1):

Spontaneous walking and circling toward the paretic side are first observed. Then, the rat, held by the tail, is placed on a rough surface and pushed gently consecutively toward the ipsi- and contralateral sides to assess resistance to push. Finally, the rat is hung by the tail, sequentially by the right and the left hand of the experimenter, and lifted above the bench to assess the body rotation and flexions of the forelimbs and hindlimbs.

Each subtest is graded on a scale from 0 to 2 (0=no response or totally abnormal response, 1=weak or abnormal response, 2=normal response). Absence of deficit is represented by a maximum neurological score of 28.

The test is performed 24 h and 48 h after surgery.

### Infarct Assessment

Animals are anaesthetized 48 hours after surgery with isoflurane and killed by decapitation. Brains are extracted and quickly frozen in isopentane solution (Sigma) at −20° C. Brains are embedded into Tissue-Tek (Raymond A Lamb Ltd, C/101.25) and cut with a cryostat. Coronal sections (20 μm), spaced 800 μm are stained with thionin (Sigma, 0.05%) during 5 minutes. The sections are scanned and infarct volumes are determined using ImageJ software (http://rsb.info.nih.gov/ij/). The volumes of infarct are corrected relative to the volume of the whole brain and of the oedema (difference between the volume of the ipsi- and contralateral hemispheres).

### Statistical Analysis

Quantitative data obtained in the neurological score will be analyzed by a 2 way ANOVA (time×treatment) followed by 1 way ANOVA (treatment) and post-hoc comparisons using unpaired Student's t test.

For the object recognition test, data will be analyzed by comparing treated groups with an appropriate control group using unpaired Student's t tests. In addition, for each group, the time spent investigating the familiar object (E2F) will be compared with the time spent investigating the novel object (E2N), and the RI will be compared with chance value (i.e. RI=0), using paired Student's t tests.

Quantitative data obtained after infarct assessment will be analyzed by comparing treated groups with an appropriate control group using unpaired Student's t tests.

### Example 15

### Pulmonary Hypertension (Ex Vivo) (Prophetic)

### Efficacy of IMD 026259 on Hypoxic Pulmonary Vasoconstriction (HPV).

By this study it can be investigated whether IMD 026259 acts inhibitory on specific regulatory mechanisms of HPV. It can be investigated whether IMD 026259 suppresses HPV by inhibiting the endogenous sensor/sensation of oxygen.

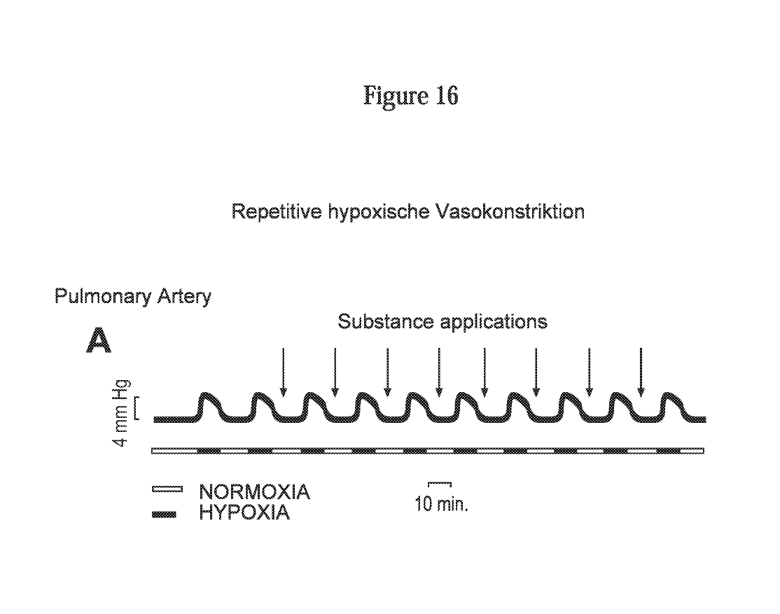

### I) Dose Response (DR) Relationship Under Acute HPV (See FIG. 16).

An effect of IMD-026259 on HPV can be compared in a non-hypoxic model of vasoconstriction. The latter can be induced by a bolus-application of Thromboxan mimetic U46619.

### Experimental Groups:

a) DR curve under repetitive HPV (see FIG. 16) (repetitive hypoxic vasoconstriction)
b) Repetitive HPV with Placebo-Application
c) DR curve with repetitive U46619 induced vasoconstriction
d) Control of repetitive U46619 application with placebo application

### II) Investigating the Effect of IMD 026259 on Protracted HPV (180 Min of Continuous Hypoxic Ventilation).

### Groups

| | |
|---|---|
| a) 180 min hypoxic ventilation mit IMD 026259 | n = 8 |
| b) 180 min hypoxic ventilation with Placebo | n = 8 |

### Approach I)

Lungs from anaesthetized mice are extracted from thorax by surgery. The lungs are perfused and ventilated under isolation. For perfusion a Krebs-Henseleit buffer is used. The lungs are ventilated with a specific gas mixture (21% O2, 5,3% CO2). Hypoxic vasoconstriction is induced by repeated hypoxic ventilation (1% O2, 5,3% CO2). The periods of hypoxic ventilation last for 10 min each, while iterated with phases of normoxic ventilation for 15 min each. Inhibitors (test items) are introduced into the perfusion media 5 min after the end of normoxic ventilation. Their effect on the strength of the HPV is quantified.

The HPV strength can be measured and indicated as an increase of pulmonary pressure (PAP; see FIG. 16 from Weissmann et al., Respir Physiol. 1995). The PAP is directly proportional to the vascular resistance, since the lungs are perfused with constant volume (Roth et al. Am J Respir Crit. Care Med 2009, in press; Weissmann et al., Proc Natl Acad Sci USA. 2006 103:19093)

### Approach II)

In this study lungs are hypoxically ventilated for more than three hours (1% O₂), to investigate the effect of a defined test item dose on protracted HPV as found by approach I (e.g. Weissmann et al. Am J Respir Cell Mol Biol: 34: 505-13, 2006).

### Example 16

### Pulmonary Hypertension (In Vivo) (Prophetic)

To determine the effect of IMD-026259 on hypoxia-induced pulmonary hypertension mice are kept under chronical hypoxia (10% O₂, normobar). Thereby, a pulmonary hypertension is developed within 3 weeks (Mittal et al., Circ Res. 2007 101:258; Circulation. 2008 118:1183).

The pulmonary hypertension within this model is quantified by determination of cardiac hypertrophy, by quantification of right-ventricular systolic pressure and vascular morphometrie.

Application of IMD-026259 is performed during hypoxia for three weeks twice daily via oral gavage at two different doses (1 and 3 mg/kg respectively).

This results in 4 experimental groups (study branches):

| | | |
|---|---|---|
| 1) | Normoxic control (3 weeks normoxia) | n = 10 |
| 2) | Hypoxia control (vehicle treatment; | n = 10 |
| | 3 weeks of hypoxia, 10% O2) | |
| 3) | Hypoxia (IMD-026259, 1 mg/kg; | n = 10 |
| | 3 weeks of hypoxia, 10% O2), | |
| 4) | Hypoxia (IMD-026259, 3 mg/kg; | n = 10 |
| | 3 weeks of hypoxia, 10% O2), | |

The study described above can answer the question whether IMD-026259 is capable to reduce or inhibit pulmonary hypertension, in particular whether by inhibition of HIF-1 the development of hypoxia-induced pulmonary hypertension can be suppressed.

1. A method for the treatment and/or prophylaxis of an angiogenesis-related disorder in a patient in need of such treatment and/or prophylaxis, said method comprising administering to said patient an effective amount thereof of a compound having the formula (I): 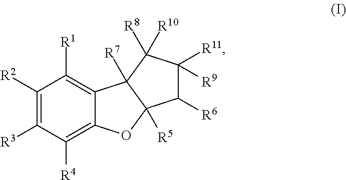
wherein
R¹, R², R³ and R⁴ independently of each other denote —H; —F; —Cl; —Br; —I; —NO₂; —CN; —OH; —O—C₁₋₈-alkyl; —O-phenyl; —O—C₁₋₈-alkyl-phenyl; —O—C(═O)—C₁₋₈-alkyl; —O—C(═O)-phenyl; —C₅₋₁₂-carbohydrate bound via one of its oxygen atoms; 6-(1,2-dihydroxy-ethyl)-3-methoxy-2-hydroxy-1,4-dioxan-2-yl;
or R¹ and R² or R² and R³ or R³ and R⁴ together with the two carbon atoms they are bound to form a five-membered ring with —O—CH₂—O— or a six-membered ring with —O—CH₂—CH₂—O—, while the other radicals R¹ to R⁴ are independently selected from those mentioned above;
R⁵ and R⁶ are phenyl;
R⁷ is —OH; —O—C₁₋₁₂-alkyl; —O-phenyl; —O—C₁₋₈-alkyl-phenyl; —O—C(═O)—C₁₋₈-alkyl; —O—C(═O)-phenyl;
R⁸ is H; —OH; —O—C₁₋₈-alkyl; —O-phenyl; —NH₂; —NH—C₁₋₈-alkyl; —N(C₁₋₈-alkyl)₂;
R⁹ is H; —C(═O)—OH; —C(═O)—O—C₁₋₈-alkyl; —C(═O)—O-phenyl; —C(═O)—C₁₋₈-alkyl; —C(═O)—O—C₁₋₈-alkyl; —C(═O)—NH₂; —C(═O)—NH—C₁₋₈-alkyl; —C(═O)—N—(C₁₋₈-alkyl)₂; or denotes —C(═O)-heterocyclyl, wherein said heterocyclyl contains at least one N-atom which is bound to the C(═O)-group;
R¹⁰ and R¹¹ are H;
or R⁸ and R¹⁰ together denote ═O, ═S, or ═NR¹⁵,
wherein R¹⁵ is —C₁₋₈-alkyl; —OH; —O—C₁₋₈-alkyl; or —O-phenyl;
or R¹⁰ and R¹¹ together form a single bond and R⁸ and R⁹ together form a group of the formula (II): 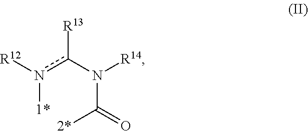
wherein
1* is the bond via R⁸ and
2* is the bond via R⁹, respectively;
the dotted line is a single or a double bond, wherein in case of a double bond R¹² does not exist;
R¹² is —H or —C₁₋₃-alkyl;
R¹³ is H; —C₁₋₈-alkyl, —OH; —O—C₁₋₈-alkyl; or —O-phenyl;
R¹⁴ is H, —C₁₋₈-alkyl;
or R¹³ and R¹⁴ together with the carbon and nitrogen atoms they are bound to form a heterocyclyl;
wherein “alkyl” in each case can be unsubstituted or substituted with one, two or three substituents independently of each other selected from the group consisting of —F, —Cl, —Br, —I, —OH, —OCH₃, —OCH₂CH₃, —O—CH₂-phenyl, —OC(═O)CH₃, —CHO, —CO₂H, —NH₂, —NH—(C₁₋₈-alkyl), —NH-(phenyl), —NH—(CH₂-phenyl), —N(C₁₋₈-alkyl)₂ and heterocyclyl, wherein said heterocyclyl contains at least one N-atom which is connected to the alkyl residue;
wherein “phenyl” in each case can be unsubstituted, or substituted with one, two or three substituents independently of each other selected from the group consisting of —F, —Cl, —Br, —I, —OH, —OCH₃, —OCH₂CH₃, —OC(═O)CH₃, —CN, —NO₂, —NH₂, —CH₃, CF₃, —CHO and —CO₂H;
or a physiologically acceptable salt thereof.

2. The method according to claim 1, wherein the angiogenesis-related disorder is selected from the group consisting of diseases of the urogenital tract.

3. The method according to claim 2, wherein the diseases of the urogenital tract are selected from the group consisting of non-inflammatory diseases of the female genital tract, endometriosis of the uterus, endometriosis of the ovary, endometriosis of tuba uterine, endometriosis of the intestine, endometriosis of scars, endometriosis of septum rectovaginale, endometriosis of the vagina, and endometriosis of the pelvis peritoneum.

4. The method according to claim 1, wherein the angiogenesis-related disorder is selected from the group consisting of eye diseases.

5. The method according to claim 4, wherein the eye diseases are selected from the group consisting of macular degeneration, vitelliform dystrophy (Best disease), retinopathies, diabetic retinopathy, glaucoma, neuroscular glaucoma, choroidal neovascularisation, occult choroidal neovascularisation, neovascularisation of the cornea, retrolental fibroplasias and rubeosis iridis.

6. The method according to claim 1, wherein the angiogenesis-related disorder is selected from the group consisting of lung diseases.

7. The method according to claim 6, wherein the lung diseases are selected from the group of consisting of airway remodelling, COPD (chronic obstructive respiratory disorder), ARDS (acute respiratory distress syndrome), infant respiratory distress syndrome, pulmonary hypertension, pulmonary sarcoidosis and idiopathic pulmonary fibrosis.

8. The method according to claim 1, wherein the angiogenesis-related disorder is selected from the group consisting of kidney diseases.

9. The method according to claim 8, wherein the kidney diseases are selected from the group of consisting of nephropathies, chronic hypoxia induced diseases, ESRD, renal fibrosis, renal artery stenosis, and glomerulonephritis.

10. The method according to claim 1, wherein the angiogenesis-related disorder is selected from the group consisting of osteoarthritis.

11. The method according to claim 10, wherein the osteoarthritis is selected from the group consisting of gonarthrosis, coxarthrosis, polyarthrosis, rhizarthrosis, and further arthroses.

12. The method according to claim 1, wherein the angiogenesis-related disorder is selected from the group consisting of rheumatic disorders.

13. The method according to claim 12, wherein the rheumatic disorders are selected from the group consisting of rheumatoid arthritis.

14. The method according to claim 1, wherein
R⁸ is H; —OH; —O—C₁₋₈-alkyl; —O-phenyl; —NH₂; —NH—C₁₋₈-alkyl; —N(C₁₋₈-alkyl)₂;
R⁹ is H; —C(═O)—OH; —C(═O)—O—C₁₋₈-alkyl; —C(═O)—O-phenyl; —C(═O)—C₁₋₈-alkyl; —C(═O)—O—C₁₋₈-alkyl; —C(═O)—NH₂; —C(═O)—NH—C₁₋₈-alkyl; —C(═O)—N—(C₁₋₈-alkyl)₂; or denotes —C(═O)-heterocyclyl, wherein said heterocyclyl contains at least one N-atom which is bound to the C(═O)-group;
R¹⁰ and R¹¹ are —H;
or R⁹ and R¹⁹ together denote ═O, ═S, or ═NR¹⁵,
wherein R¹⁵ is —C₁₋₈-alkyl; —OH; —O—C₁₋₈-alkyl; or —O-phenyl.

15. The method according to claim 1, wherein
R¹, R², R³ and R⁴ independently of each other denote —H; —F; —Cl; —Br; —I; —NO₂; —CN; —OH; —O—C₁₋₈-alkyl; —O-phenyl; —O—C₁₋₈-alkyl-phenyl; —O—C(═O)—C₁₋₈-alkyl; —O—C(═O)-phenyl;
R⁵ and R⁶ are phenyl;
R⁷ and R⁸ are independently of each other —OH, or —O—C₁₋₈-alkyl;
R⁹, R¹⁰ and R¹¹ are —H.

16. The method according to claim 1, wherein
R¹ and R³ independently of each other denote H; —OH; —O—C₁₋₈-alkyl; —O-phenyl; —O—C₁₋₈-alkyl-phenyl; —O—C(═O)—C₁₋₈-alkyl; —O—C(═O)-phenyl;
with the proviso that at least one of the radicals R¹ and R³ is not —H;
R² and R⁴ are H;
R⁵ and R⁶ are phenyl,
R⁷ and R⁸ are independently of each other —OH, or —O—C₁₋₈-alkyl;
R⁹, R¹⁰ and R¹¹ are —H.

17. The method according to claim 1, wherein
R¹ and R³ independently of each other denote —H; —OH; —O—C₁₋₈-alkyl; O-phenyl; —O—C₁₋₈-alkyl-phenyl;
with the proviso that at least one of the radicals R¹ and R³ is not —H;
R² and R⁴ are —H;
R⁵ and R⁶ are phenyl,
R⁷ and R⁸ are —OH;
R⁹, R¹⁰ and R¹¹ are —H.

18. The method according to claim 1, wherein the compound has the formula (Ie): 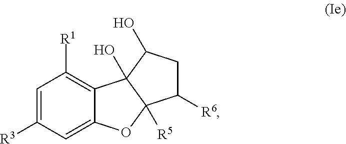
wherein
R¹ denotes —H; unsubstituted or substituted with one substituent selected from the group consisting of —OCH₃, —OCH₂CH₃, —NH₂, —NH(CH₃), —NH(CH₂CH₃), —N(CH₃)₂, —N(CH₂CH₃)₂, 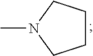
—O—CH₂-phenyl, unsubstituted;
R³ denotes —OH; —O-phenyl, unsubstituted; —O—C₁₋₈-alkyl, unsubstituted or substituted with one substituent selected from the group consisting of —F, —Cl, —OCH₃, —OCH₂CH₃, —NH₂, —NH(CH₃), —NH(CH₂CH₃), —NH(CH(CH₃)₂), —NH(C(CH₃)₃), —NH(CH₂-phenyl) or —NH(phenyl), wherein phenyl in each case is unsubstituted, —N(CH₃)₂, —N(CH₂CH₃)₂, 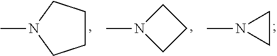
—O—CH₂-phenyl, unsubstituted;
R⁵ and R⁶ are phenyl, unsubstituted, or substituted with one, two or three substituents independently of each other selected from the group consisting of —F, —Cl, —Br, —I, —OH, —OCH₃, —NH₂, —CH₃ and —CF₃.
